(19) United States
(12) Patent Application Publication
(10) Pub. No.: US 2026/0014093 A1
(43) Pub. Date: Jan. 15, 2026

(54) ATOMIC LAYER DEPOSITION (ALD) PROCESSES APPLIED TO PHAGE AND PHAGE-LIKE PARTICLE PLATFORM YIELD THERMOSTABLE THERAPEUTIC AGENTS

(71) Applicant: THE REGENTS OF THE UNIVERSITY OF COLORADO, A BODY CORPORATE, Denver, CO (US)

(72) Inventors: Theodore W. RANDOLPH, Denver, CO (US); Alyssa WITEOF, Denver, CO (US); Qin YANG, Denver, CO (US); Carlos CATALANO, Denver, CO (US)

(21) Appl. No.: 19/273,219

(22) Filed: Jul. 18, 2025

Related U.S. Application Data
(63) Continuation of application No. PCT/US2024/012662, filed on Jan. 23, 2024.
(60) Provisional application No. 63/481,182, filed on Jan. 23, 2023.
(60) Provisional application No. 63/482,010, filed on Jan. 27, 2023.

(51) Int. Cl. A61K 9/51; C12N 7/00; C23C 16/44; C23C 16/455

(52) U.S. Cl. CPC A61K 9/5184; C12N 7/00; C12N 2795/00023; C12N 2795/00051; C23C 16/4417; C23C 16/45555

(57) ABSTRACT

Embodiments of the present disclosure provide novel compositions and methods for making and using thermostable bacteriophage or bacteriophage-derived phage-like-particle (PLP)-containing formulations. In certain embodiments, compositions and methods are disclosed for embedding, decorating and/or associating at least one antigen or agent, or bioactive molecule on the surface of the bacteriophage or PLPs. In accordance with these embodiments, bacteriophage or PLPs harboring one or more antigen or agent, or bioactive molecule can further be thermostabilized and/or coated with one or more atomic layer deposition applied coating layer for control or timed release of the one or more antigen or agent when administered to a subject.

### CROSS-REFERENCED APPLICATIONS

This Continuation Application claims priority to International Application PCT/US2024/012662 filed Jan. 23, 2024, which claims priority to U.S. Provisional Application No. 63/482,010 filed Jan. 27, 2023, and U.S. Provisional Application No. 63/481,182 filed Jan. 23, 2023. These applications are incorporated herein by reference in their entireties for all purposes.

### FIELD

Embodiments of the present disclosure provide novel compositions and methods for making and using thermostable bacteriophage or bacteriophage-derived phage-like-particle (PLP)-containing formulations. In certain embodiments, compositions and methods are disclosed for embedding or associating at least one antigen, immunogenic agent or other therapeutic agent, or bioactive molecule to a surface of PLPs. In accordance with these embodiments. PLPs harboring one or more antigen, agent, or bioactive molecule can further be thermostabilized and/or coated with one or more coating layer for control release of the at least one antigen, immunogenic agent or other therapeutic agent, or bioactive molecule when administered to a subject to treat, reduce onset of, or prevent a health condition.

### BACKGROUND

Therapeutic impacts of vaccines and other therapeutic treatments have many challenges. One challenge is the storage and/or delivery of a vaccine or time-release delivery of a bioactive agent to a subject. For example, multiple administrations over various periods of time are often required to provide successful immunity, which can decrease the likelihood that a subject will take the necessary steps to obtain a required second or more administrations. In addition, therapeutic agents, vaccines, and vaccine formulations often exhibit increased instability during storage, transport, and handling. Further, certain vaccines against a particular pathogen must be separately administered at the same time or at different times to a subject to avoid being ineffective when combined or administered at the same time as another vaccine against another pathogen. Additionally, therapeutic agents, vaccines and vaccine formulations are typically associated with high manufacturing costs. which can limit their availability to those in need. Therefore, a need exists for improved therapeutic agent and vaccine transport, storage and delivery.

### SUMMARY

Embodiments of the present disclosure provide novel compositions and methods for making and using thermostable bacteriophage or bacteriophage-derived phage-like-particle (PLP)-containing formulations, for example, PLP designer particle formulations. In certain embodiments, compositions and methods are disclosed for embedding or associating at least one antigen, immunogenic agent or other therapeutic agent, or bioactive molecule to or onto the surface of PLPs. In accordance with these embodiments, PLPs harboring or carrying at least one antigen. immunogenic agent or other therapeutic agent, or bioactive molecule can further be thermostabilized and/or coated with one or more atomic layer deposition (ALD) applied coating layer for controlled release of the at least one antigen, immunogenic agent or other therapeutic agent, or bioactive molecule when administered to a subject.

In other embodiments, the PLP-designer particles can increase stability and/or compatibility of the at least one antigen, immunogenic agent or other therapeutic agent, or bioactive molecule and/or provide for timed-release delivery of the at least one antigen, immunogenic agent, or other therapeutic agent, or bioactive molecule. In some embodiments, primary and boost doses of PLP-designer particles harboring or at least one antigen, immunogenic agent or other therapeutic agent, or bioactive molecule or the like can be administered to a subject in a single administration. In other embodiments, PLP-designer particles can include antigens or therapeutic agents against two or more pathogens either in the same or in separate particles; one or more agents to target one or more cellular pathways or to enhance one or more pathways to treat, prevent or reduce onset of a health condition (e.g., cancer, an infection or acute or chronic condition).

Certain embodiments provide compositions and methods for making PLPs thermostable particles carrying or harboring at least one antigen, immunogenic agent or other therapeutic agent, or bioactive molecule. In accordance with these embodiments, the methods can include combining at least one PLP with at least one antigen, immunogenic agent or other therapeutic agent, or bioactive molecule where the at least one antigen, immunogenic agent or other therapeutic agent, or bioactive molecule is affiliated or bound to the PLP. Introducing the at least one antigen, immunogenic agent or other therapeutic agent, or bioactive molecule associated with the PLP to at least one glass-forming agent in a solution to form a primary liquid composition. Essentially drying the primary liquid composition by lyophilizing, spray-drying, spray-freeze-drying, vacuum-drying, or other method, the primary liquid composition to form the at least one antigen, immunogenic agent or other therapeutic agent, or bioactive molecule affiliated with the PLP into essentially dry glassy particles. In certain embodiments, the methods can include introducing the at least one antigen, immunogenic agent or other therapeutic agent, or bioactive molecule to the PLP before, during, or after essentially drying the PLP and optionally, coating the PLP agent, antigen or bioactive macromolecule-containing glassy microparticles with one or more coating layers using ALD. In certain embodiments, the primary liquid composition can be spray-dried to conserve molecular structures and to form improved glassy particles in preparation for storage and/or coating of load-bearing PLP particles. In other embodiments, essentially dried PLP particles (post spray-drying) can be reconstituted or stored for a predetermined period and then reconstituted and subsequently decorated with one or more antigen, immunogenic agent or other therapeutic agent, or bioactive molecule. In accordance with these embodiments, the essentially dried particles can be prepared in bulk production and stored as a single lot or aliquoted into smaller concentrations and stored. In other embodiments, the one or more antigen, immunogenic agent or other therapeutic agent, or bioactive molecule can be specifically selected at a time of use to decorate the essentially dried particles (e.g., without decoration) to reduce onset of, prevent or treat a health condition.

In accordance with these embodiments, the at least one antigen, therapeutic agent, or bioactive molecule can include an immunogenic agent or antigen, for example a viral antigen, a bacterial antigen, a fungal antigen, a toxin, or a combination thereof. In some embodiments, the at least one antigen, therapeutic agent, or bioactive molecule can include but is not limited to, a polynucleotide (e.g., RNA, DNA, mRNA, an siRNA, hybrid polynucleotide, substituted polynucleotide, or the like), or chimera, a recombinant peptide, a recombinant protein, one or more peptides derived from target proteins or pathogens, synthetic peptides or proteins, virus-like particles, live virus, live, attenuated virus, inactivated virus, or a mixture, complex or combination thereof.

In certain embodiments, the at least one antigen, therapeutic agent, or bioactive molecule can include an agent or bioactive molecule that modulates such as inhibits or activates a pathway such as a pathway for modifying or treating a health condition. In accordance with these embodiments, the health condition can include, but is not limited to, cancer, inflammatory condition, autoimmune disorder, or combination thereof. In other embodiments, PLP agent- or antigen-containing particles can be coated (e.g., ALD-coated) or uncoated and administered to a subject for treating, preventing or reducing the onset of a health condition including, but is not limited to, cancer, an inflammatory condition, and/or an autoimmune disorder.

In other embodiments, the at least one agent can include one or more antigens for example, antigens derived from any pathogenic virus or pathogenic bacteria, fungi, protozoa, prion, toxoid, or the like. In some embodiments, the one or more antigen or agent is derived from corona virus, SARS, human papilloma virus (HPV), ricin toxin, Bacillus anthracis, Clostridium botulinum, Ebola virus, poliovirus, norovirus, rotavirus, hepatitis C, varicella, herpes simplex, cytomegalovirus, Japanese encephalitis, dengue virus or other flavivirus, West Nile virus, Zika virus, Chikungunya virus or other pathogenic alpha virus, Yersinia, Pneumococcus, Salmonella, Clostridium difficile, or a combination thereof. In other embodiments, the at least one agent can be a multimeric complex, for example, a viral or bacterial complex representing one, two, three or more pathogens.

In some embodiments, the at least one glass-forming agent or at least one disaccharide can include at least one of trehalose, sucrose, ficoll, dextran, hydroxyethyl starch, polyvinylpyrrolidone (PVP), maltose, lactulose, maltotriose, lactose, mannitol, glycine, mannitol and glycine, cyclodextrin, povidone, a combination thereof or the like. In certain embodiments, the at least one glass-forming agent can include trehalose or sucrose. In accordance with these embodiments, a glass-forming agent can be present in the primary liquid composition in a weight-to-volume (w/v) concentration of from about 0.1% to about 40.0%, from about 1.0% to about 30.0%, from about 1.0% to about 20.0%, from about 5.0% to about 20.0% or from about 8.0% to about 20.0% (w/v) or from about 8.0% to about 15.0%.

In other embodiments, the glass-forming agent can further include at least one smoothing excipient. In accordance with these embodiments, the smoothing excipient can be, but is not limited to, a hydroxyethyl starch or other pharmacologically acceptable plasma expander such as human serum albumin (HSA), other serum albumins, dextran, hetastarch, plasma protein factor and the like, or a combination thereof. In certain embodiments, the smoothing excipient can also function as a primary glass-forming agent. In some embodiments, the smoothing excipient is hydroxyethyl starch. In some embodiments, a smoothing agent disclosed herein can be the primary glass-forming agent. In accordance with these embodiments, the smoothing excipient acting as a smoothing agent to smooth surface of PLP particles disclosed herein can be present in the primary composition at a weight-to-volume (w/v) concentration from about 0.1% to about 40.0%, from about 1.0% to about 30.0%, from about 5.0% to about 20.0%, or from about 8.0% to about 15.0%. In certain embodiments, the smoothing excipient can be different than the one or more glass-forming agents, and the smoothing excipient can be present in the primary composition in a weight-to-volume (w/v) concentration from about 0.1% to about 10%, from about 0.1% to about 5%, from about 0.1% to about 2.5%, from about 0.1% to about 0.5%. In certain embodiments, the glass-forming agent present in the composition can be sucrose and/or trehalose and the smoothing excipient for the PLP particles can be hydroxyethyl starch. In some embodiments, the glass-forming agent present in the composition is trehalose and the smoothing excipient for the phage or PLP particles is hydroxyethyl starch.

In some embodiments, each layer of one or more coating layers applied by ALD coating to one or more loaded essentially dry PLP particle (e.g., loaded as used herein can mean one or more antigen or agent bound to or affiliated with the essentially dry PLP particles, loaded PLP particles) can include, but is not limited to, metallo-organic material, a metal oxide, or a metal alkoxide or the like. In some embodiments, the coating material can include, but is not limited to, aluminum oxide (Al₂O₃). an aluminum alkoxide (e.g., alucone), silicon dioxide (SiO₂). titanium dioxide (TiO₂), zinc dioxide (ZnO₂) or silicon nitride (Si₃N₄) alone or in a suitable combination composition or alternating or other pattern of layering such as 1:2. 1:3, 1:4. 1:5 or other pattern (e.g., alternating layers of aluminum agents with silicon- or titanium-containing compositions. for example). In accordance with these embodiments, the ALD coating layer(s) can be about 0.1 nm to about 20 nm in thickness. In certain embodiments. thermostable PLP-containing antigen or agent particles (loaded PLP particle) can include a number of coating layers sufficient to delay release or provide a timed-release of or prolong storage of the at least one antigen and/or agent from the loaded PLP particle. In accordance with these embodiments. ALD coating layers contemplated herein can completely encase loaded thermostable PLP particles. In other embodiments. these ALD-applied coating layers can be without pinholes or the like.

In certain embodiments, the one or more coating layer(s) or ALD coating layer(s) disclosed herein can serve or also serve as an adjuvant to enhance an immune response in a subject against one or more antigen or agent(s) of the PLP-containing particles derived from one or more pathogen or other therapeutic agent. In certain embodiments, the one or more coating layer(s) or ALD coating layer(s) disclosed herein can contain a coating layer concentration capable of inducing a rapid immune response to the one or more antigen or agent(s) of the PLP particles prior to, or simultaneously with dissolution and release of the one or more antigen or agent from the PLP particles.

In other embodiments, the antigen or agent-containing PLP particle can further include at least a second antigen or agent deposited as a layer on an outermost coating layer of a loaded PLP particle. In accordance with these embodiments, the layer of the at least the second agent or antigen can be embedded in the coated PLP of at least a second glass-forming agent to stabilize the second antigen or agent. In certain embodiments, the at least the second agent or antigen can be further coated with at least one coating layer by ALD or other coating method.

In some embodiments, uncoated or coated PLP antigen or agent-containing thermostable particles described herein can be stored without refrigeration temperatures of up to about 50° C. to about 60° C. for extended periods of time. In certain embodiments, uncoated or coated PLP antigen or agent-containing thermostable particles described herein can be stored without refrigeration up to about 50° C. to about 60° C. up to about 1 day, to about 3 days. to about one week, to about 2 weeks, to about 1 month, to about 2 months, to about 3 months, or up to about 4 months, or up to about 6 month, or up to about 9 months, or up to about 12 months, or up to about 15 months, or up to about 18 months, or up to about 24 months or longer without negative effect on the uncoated or coated PLP antigen or agent-containing thermostable particles (e.g. degradation)

Other embodiments provide for compositions including a plurality of uncoated, coated or ALD-coated PLP antigen or agent-containing thermostable particles described herein. In certain embodiments,these compositions can include PLP antigen- or agent-containing particles or coated particles in a pharmaceutically acceptable excipient to create a pharmaceutically acceptable formulation or composition. In other embodiments, these compositions can include ALD-coated PLP antigen- or agent-containing particles in a pharmaceutically acceptable excipient to create a pharmaceutically acceptable formulation or composition—In accordance with these embodiments, formulations or compositions described herein are capable of eliciting an immune response or response to the antigen or agent associated or bound to and released from the PLP when administered to a subject and exposed to the subject by degradation or dissolution of the PLP particle and/or coating layers over time.

In some embodiments, the formulation or composition or pharmaceutical composition is a single-administration composition of a prime dose and boost dose of at least one antigen or agent affiliated with or bound to coated or uncoated PLP particles. In accordance with these embodiments, the prime and boost doses of the at least one antigen or agent can be in the same coated or uncoated agent-containing PLP particle, or in separate coated or uncoated PLP particles. When in separate particles, the priming dose can be sequestered in a particle without any coating layers, or in particles, while the boost dose is in another particle such as a coated PLP particle.

In some embodiments, uncoated or coated antigen or agent affiliated PLP particle-containing compositions and formulations disclosed herein can be used to treat a condition or prevent onset of a condition such as a health condition. In other embodiments, compositions and formulations disclosed herein can contain a standard vaccine or treatment for a condition as a combination therapy or prophylactic.

Yet other embodiments provide for kits that can include at least one type of coated or uncoated PLP antigen- or agent-containing particles described herein and at least one container or components for creating such a composition. In certain embodiments, the kits include components for making the compositions including coating agents for use in ALD coating of thermostable PLP agent, antigen, or bioactive molecule-containing particles.

### BRIEF DESCRIPTION OF THE FIGURES

The accompanying drawings are incorporated into and form a non-limiting part of the specification to illustrate several examples of the present disclosure.

FIG. 1A is a schematic representation of phage PLP generation and loading with one or more antigen or therapeutic agent (decoration) of some embodiments of the present disclosure.

FIG. 1B is a schematic representation of thermostabilization of phage PLP particles and subsequent coating by ALD processes of some embodiments of the present disclosure.

FIG. 1C is a schematic representation of various incubations of formulations disclosed herein and administration to a test animal for assessing immunological responses in PLP particles disclosed herein according to some embodiments of the present disclosure.

FIG. 2 illustrates a representative plot of stability of formulated phage. Phage λ was incubated at room temperature (green lines) or 37° C. (red lines) as either liquid (circles) or spray dried powders (diamond) formulations. Phage λ maintained infectivity in all formulations and at all incubation temperatures tested except for the liquid formulation incubated at 37° C., which by one year had no detectable titer (depicted as ND on the graph). according to some embodiments of the present disclosure.

FIG. 3A includes some representative images demonstrating some biophysical characterization of spray dried and ALD-coated loaded-PLPs after incubation at different temperatures. A.) represents SEM images of spray dried particles (middle row, left columns) or ALD-coated particles (bottom row, left columns) incubated at 4° C. (left) or 50° C. (right). TEM images of liquid loaded-PLPs incubated at 4° C. (left) or 50° C. (right) resuspended spray dried particles (middle row) or ALD-coated particles (bottom row) incubated at 4° C. (left) or 50° C. (right).

FIG. 3B represents various size distributions determined for loaded-PLPs in liquid suspensions prior to spray drying (solid line, B (i.)) and in reconstituted spray dried powder after spray drying (dashed line, i.). Liquid loaded-PLP formulation incubated at 4° C. (solid line, B (ii)) or 50° C. (dashed line. B (ii)). Spray dried loaded-PLPs formulation incubated at 4° C. (solid line, B (iii)) or 50° C. (dashed line, B (iii)). ALD-coated loaded-PLP formulations incubated at 4° C. (solid line, B (iv)) or 50° C. (dashed line, B (iv)). according to some embodiments of the present disclosure.

FIGS. 4A-4D are representative plots on log scale depicting antibody responses in an animal models administered various particles disclosed herein incubated for a month at 4° C. or 50° C. Serum samples were collected at time points after injection, and antibody titers against loaded-PLPs (panels 4A and 4B) and control samples (panels 4C and 4D) were measured according to some embodiments of the present disclosure.

FIGS. 5A-5B are images of representative gel electrophoresis illustrating successful decoration or loading of PLPs disclosed herein. 5A) represents an SDS-PAGE gel illustrating wild type PLPs (WT) in lane 1 and PLPs decorated with cargo in lane 2. 5B) illustrates a native agarose gel illustrating undecorated or un-loaded PLPs in lane 1 and PLPs decorated or loaded in lane 2. Undecorated PLPs have two distinct bands whereas decorated or loaded PLPs have one larger band according to some embodiments of the present disclosure.

FIG. 6 is a graphical representation of phage stability in essentially dry. glassy powders prepared by spray drying solutions disclosed herein. Phage activity can be expressed as plaque-forming units, measured against plated cultures of the targeted bacteria. Powder samples were stored at various temperatures for prolonged periods. Titer losses less than 1 log were observed after prolonged storage at the various temperatures of some embodiments of the present disclosure.

FIG. 7 is a graphical representation of phage stability in essentially dry, glassy powders prepared by spray drying solutions and then ALD coated as disclosed herein. Phage activity can be expressed as plaque-forming units, measured against plated cultures of the targeted bacteria. Powder samples were stored at various temperatures for prolonged periods. Titer losses less than 1 log were observed after prolonged storage at the various temperatures of some embodiments of the present disclosure.

FIG. 8 is a graphical representation of phage stability in essentially dry, glassy powders prepared by spray drying solutions disclosed herein. Phage activity can be expressed as plaque-forming units, measured against plated cultures of the targeted bacteria. Powder samples were stored at various temperatures for prolonged periods. Titer losses less than 1 log were observed after prolonged storage at the various temperatures of some embodiments of the present disclosure.

FIG. 9 is a graphical representation of phage stability in essentially dry, glassy powders prepared by spray drying solutions and then ALD coated as disclosed herein. Phage activity can be expressed as plaque-forming units, measured against plated cultures of the targeted bacteria. Powder samples were stored at various temperatures for prolonged periods. Titer losses less than 1 log were observed after prolonged storage at the various temperatures of some embodiments of the present disclosure.

FIG. 10 is a graphical representation of phage stability in essentially dry. glassy powders prepared by spray drying solutions disclosed herein. Phage activity can be expressed as plaque-forming units, measured against plated cultures of the targeted bacteria. Powder samples were stored at various temperatures for prolonged periods. Titer losses less than 1 log were observed after prolonged storage at the various temperatures of some embodiments of the present disclosure.

FIG. 11 is a graphical representation of phage stability in essentially dry, glassy powders prepared by spray drying solutions and then ALD coated as disclosed herein. Phage activity can be expressed as plaque-forming units, measured against plated cultures of the targeted bacteria. Powder samples were stored at various temperatures for prolonged periods. Titer losses less than 1 log were observed after prolonged storage at the various temperatures of some embodiments of the present disclosure.

### DETAILED DESCRIPTION

In the following sections, various exemplary compositions and methods are described to detail various embodiments. It will be obvious to one skilled in the art that practicing the various embodiments does not require the employment of all or even some of the specific details outlined herein, but rather that concentrations, times, and other specific details may be modified through routine experimentation. In some embodiments, well known methods or components have not been included in the description.

Impact of vaccines and other therapeutic agents can be limited by logistical obstacles associated with multiple dose regimens, pathogen variants, and challenges imposed by requirements for maintaining vaccines or other therapeutic agents at low temperatures during shipping, storage, and delivery to a subject. Therefore, there is a need for vaccines and other therapies that can be flexibly modified to address evolving pathogen landscapes and changes, are stable outside of narrow cold-chain temperature storage and require administration of only single doses to improve compliance. Disclosed herein is a therapeutic or vaccine platform that addresses these impediments to more widespread use. The platform relies on bacteriophage-derived phage-like-particles (PLPs) that utilize a plug-and-play antigen or therapeutic agent delivery system that allows for fast, easy alteration or substitution of antigens or therapeutic agents on the surface of the PLPs. Thermostability of PLP-based associated agents can be achieved by embedding or glassifying PLPs within glassy particles produced by lyophilization or spray-drying, and application of nanoscopic metallo-organic material, metal oxide or metal alkoxide layers applied using atomic layer deposition (ALD) for control release of the agents in vivo, yielding formulations that elicit strong responses (e.g., immune, or therapeutic responses) after administration of single doses.

Vaccines for the prevention and treatment of various health conditions provide benefits to human health but suffer from drawbacks. Prominent among drawbacks of various vaccines is a need to maintain temperatures of vaccines under tightly controlled cold-chain conditions during storage and transportation, and requirements that multiple doses of many vaccines must be administered to generate effective immune responses. These requirements complicate logistics of delivery and administration, especially in developing countries where lack of cold-chain infrastructure and limited access to medical services is problematic. Compliance with multi-dose schedules for vaccines is known to be low and studies have shown individuals are more likely to be vaccinated if there is only one dose is needed. Furthermore, as the recent COVID-19 pandemic has demonstrated, there is a need for vaccine platforms that not only are tolerant to relaxed cold-chain requirements and provide single-shot dosing, but that also enable rapid responses to emerging and evolving pathogen adaptations and changes by allowing facilitated development and modification of vaccines to accommodate and address these rapid changes.

Bacteriophages (phages) are viruses that infect bacteria. They are non-infectious to humans and are essentially non-toxic to eukaryotic cells. Emergence of multi-drug resistant bacteria has led to renewed interest in phage therapy as an alternative and/or adjunct to chemical antibiotics. Further, phage-based nanoparticles have emerged as vehicles for targeted drug delivery of anti-cancer drugs, and as nanoparticles that display antigens for vaccine development.

Bacteriophages offer numerous advantages for vaccines: they have defined shapes and sizes that allow for symmetric and defined presentation of antigenic surface ligands, are soluble in aqueous media, and can be economically prepared in large scale industrial processes for later use. Bacteriophages are currently produced in large scale for use in the food industry, and recent reviews have discussed the economic advantages of various high-titer, continuous bacteriophage manufacturing processes and some of their pharmaceutical applications. Here a phage-based platform (e.g., 2 phage or any other bacteriophage) that can flexibly deliver a wide variety of antigens and therapeutic agents in a “plug and play” fashion was developed. When co-expressed in E. coli. the A major capsid and scaffolding proteins self-assemble into icosahedral shells. forming phage-like particles (PLPs) that can be isolated in high yield. The shell surfaces of these PLPs can be decorated with a wide variety of protein, peptide, carbohydrate, and synthetic ligands via a linking agent (e.g., gpD, (a phage λ capsid decoration protein gpD)) or a decoration permitting protein. Antigens, therapeutic agents, and adjuvants coupled to gpD can be used to decorate or load a PLP particle surface alone and in combination at defined surface densities, to afford about 140 trimer spikes symmetrically displayed on the 60 nm shell surface of PLPs. Further, the symmetrically-arranged gpD protein provides additional stabilization of bacteriophage structures with other vaccines that rely on viral structures to efficiently present epitopes in repetitive arrays, the highly repetitive decoration of the surface of PLPs can serve to increase therapeutic (e.g., immune) responses for example, by stimulating B cells in a T-independent fashion.

To further develop and manufacture an effective therapeutic (e.g., vaccine), stabilizing formulations must be designed that provide for efficient storage and distribution. Thermostabilization of vaccines and other therapeutic agents can be achieved by embedding antigens, therapeutic agents, and adjuvants within glassy matrices such as organic matrices. High viscosities typical of these matrices limit molecular mobility of antigens, inhibiting or eliminating kinetics of many physical degradation pathways. Similarly, low water activity within the matrices can limit chemical damage. Lyophilization is most often used to formulate vaccines as dry glasses/matrices. Lyophilized formulations of the meningococcal A vaccine allowed relaxed cold-chain requirements that facilitated administration of the vaccine in Africa and lyophilized human papilloma virus (HPV) vaccines were demonstrated to be stable for at least 3 months at 50° C. Lyophilization is associated with potentially damaging stresses and relatively high costs, although many of these obstacles can be overcome with judicious choice of operating parameters and formulation excipients. Spray-drying is an alternative technique for formulating antigens or other agents within glassy powders that offers advantages of lower operating costs compared to lyophilization, avoiding potentially damaging ice-water interfaces. and creates fine, spherical powders that can be delivered by different routes of administration. In certain embodiments disclosed herein, loaded PLPs can be either lyophilized or spray-dried to create essentially dry formulations.

In other embodiments, atomic layer deposition (ALD) can be used to deposit nanoscopic layers of metal oxides or metal alkoxides on essentially dry powders disclosed herein. In accordance with these embodiments, particles can be coated in a fluidized bed reactor by alternating injections of gas-phase reagents (e.g, alternating trimethylaluminum and water) to deposit single molecular layers of metallo-organic material, metal oxides or metal alkoxides in self-limiting reactions. Multiple cycles of alternating injections can be used to grow uniform metal oxide or metal alkoxide layers of arbitrary, controlled thickness on particle surfaces. In certain embodiments, metal oxide layering by ALD can include layering or coating of alumina oxides.

In some embodiments, it was hypothesized that combining a tunable PLP system with spray drying and ALD coating could result in a platform with the potential to produce rapidly adaptable vaccines and other therapeutic agents that would be thermally stable and deliverable in a single shot administration dose. In accordance with these embodiments, stability of liquid suspensions of phage (e.g., λ phage) were first compared against spray dried formulations by measuring phage (e.g., λ phage) infectivity by plaque assays after incubation for one year at room temperature or 37° C. Then, phage derived PLPs (e.g., λ phage, phage VPE25, phage 80a etc.) with cargo/antigen as model antigen (See for example. the figures). These antigen decorated PLPs can be spray-dried to form glassy particles, then coated with nanoscopic layers of metal oxide in an ALD reactor (e.g., fluidized ALD reactor bed), as disclosed herein. Physical stability of PLPs can be measured by, for example, dynamic light scattering (DLS). scanning electron microscopy (SEM), and transmission electron microscopy (TEM).

In certain embodiments, bacteriophages (phages) were stabilized by spray drying to form powders or essentially dry particles that can be incubated at 37° C. or up to 60° C. for up to a year without loss of infectious activity. In some embodiments. PLPs derived from bacteriophage (e.g., λ phage, phage VPE25, phage 80α, etc.) were expressed and purified from bacterial cultures (e.g., E. coli cultures), and an in vitro conjugation strategy can be used to decorate or load specific PLP surface sites with an antigen, biologic, therapeutic agent, and/or fragment thereof (e.g., T4-lysozyme, a model antigen). In accordance with these embodiments, conjugate complexes (e.g, T4-lysozyme:PLP, Lys-PLPs, antigen, biologic, therapeutic agent, and/or fragment thereof-decorated PLPs or phage) formed can further be embedded in glassy dry powders (e.g., disaccharide-containing agent powders) formed by spray drying. In certain embodiments, essentially dry conjugate complexes of PLPs (e.g., from spray drying) can be coated with nanometer-thick layers of metallo-organic, metal oxide and/or metal alkoxide compositions deposited by ALD in a fluidized bed reactor (e.g., ALD applied alumina coating).

In some embodiments, metallo-organic, metal oxide and/or metal alkoxide coated compositions disclosed herein are stable for about 1 week to about one year or more at about 40° C. to about 60° C. In certain embodiments, the metallo-organic, metal oxide and/or metal alkoxide coatings can be designed to coat a target PLP with sufficient layers for a specific time to be released to treat, reduce onset of, or prevent a health condition. In some embodiments, as an example, alumina-coated decorated PLPs can be stable for a least a month to about 1 year at 50° C. In accordance with these embodiments, a single dose of the alumina-coated decorated PLPs elicited immune responses that were indistinguishable from responses generated by conventional two-dose, prime-and-boost dosing regimens of alum-adjuvanted decorated-PLP vaccines meaning that the alumina-coated decorated PLPs were stabilized and retained immunogenicity compared to controls.

Embodiments of the present disclosure provide novel compositions and methods for making and using thermostable bacteriophage or bacteriophage-derived phage-like-particle (PLP)-containing formulations. In some embodiments, a bacteriophage or phage can be a duplodnaviria virus that infects and replicates within bacteria and archaea. It is known that bacteriophages are composed of proteins that encapsulate a DNA or RNA genome and can have structures that are either simple or elaborate. Their genomes can encode as few as four genes (e.g., MS2) and as many as hundreds of genes. Phages replicate within the bacterium following the injection of phage genomes into its cytoplasm. Bacteriophages are ubiquitous viruses, found wherever bacteria exist. It is estimated there are more than 1000 bacteriophages exist on the planet. Phages have been used since the late 20th century as an alternative to antibiotics. They are seen as a possible therapy against multi-drug-resistant strains of many bacteria (see for example. phage therapy). Phages are known to interact with the immune system both indirectly via bacterial expression of phage-encoded proteins and directly by influencing innate immunity and bacterial clearance. In accordance with these embodiments, it has been demonstrated herein that it is possible to thermostabilize any phage (and alternatively decorate a phage for further efficacy) and each phage can be used as potential antibiotic alternatives active against infections of their respective bacterial targets (e.g., E. coli. E. faecalis, and S. aureus and others).

In certain embodiments, PLPs can include any PLP derived from a phage. In some embodiments, a PLP can be derived from a bacteriophage. Any bacteriophage is contemplated of use herein. In certain embodiments, the bacteriophage is an enveloped or a non-enveloped bacteriophage. In other embodiments, the bacteriophage and PLPs derived therefrom target and kill pathogenic bacteria.

In other embodiments, the at least one agent, therapeutic agent, biologic, and/or antigen can include one or more agent(s) or antigen(s), for example an agent derived from a pathogenic organism. In other embodiments, the at least one agent, therapeutic agent, biologic, and/or antigen can include one or more of a viral antigen, a bacterial antigen, a toxin, a prion, yeast, a fragment or subunit thereof, a chemical agent, a small molecule, an anti-cancer agent, an anti-inflammatory agent, an anti-autoimmune agent, a polynucleotide, a polysaccharide, an antibody or fragment thereof, a peptide or protein thereof, or a combination thereof. In some embodiments, the at least one agent or antigen can include one or more agent(s) or antigen(s) including, but is not limited to, a recombinant peptide, a recombinant protein, a peptide derived from a target protein or pathogen, a polysaccharide derived from a target pathogen, a synthetic peptide or protein, a virus-like particle, a live virus, a live, attenuated virus, an inactivated virus, an antigen attached to, associated with, or expressed on the surface of a virus or a bacteriophage or a combination thereof. In some embodiments, decorated or undecorated phage as disclosed herein can be used as therapy to improve treatment options for soft tissue and/or bone infections such as acute or chronic soft tissue and/or bone infections (e.g., after injury, as a side effect to a treatment and/or surgery such as reconstructive surgery). In yet other embodiments, the at least one agent, biologic, therapeutic agent, or antigen (e.g., immunogenic agent) can include one or more of a polynucleotide, a polypeptide encoded by a polynucleotide, a polypeptide, a carbohydrate, a polysaccharide, or the like. In accordance with these embodiments, a polynucleotide can include, but is not limited to, DNA, RNA, mRNA, siRNA, other polynucleotide, a chimeric molecule thereof or fragment or subunit thereof. In certain embodiments, a chimera can include a combination of at least one polynucleotide segment and at least one polypeptide segment or a mixture of polynucleotide segments and/or polypeptide segments or other chimeric molecule. In some embodiments, the polynucleotide can include an mRNA encoding a full length, or fragment or subunit of a targeted agent (e.g., virus or bacteria or other pathogenic microorganism).

In certain embodiments, agent, biologic, therapeutic agent, or antigen (e.g., immunogenic agent) contemplated herein can include an agent derived from at least one pathogen and then complexed with or decorating PLPs disclosed herein and then spray-dried and/or coated by ALD processes as disclosed herein for improved stability for transport or administration via the PLP vehicle. In some embodiments, the pathogen can include a virus. In certain embodiments, the pathogenic virus can be, for example, a papovavirus (e.g., papillomaviruses, including human papilloma virus (HPV)), a herpesvirus (e.g., herpes simplex virus, varicella-zoster virus, bovine herpesvirus-1, cytomegalovirus), a poxvirus (e.g., smallpox virus), a reovirus (e.g., rotavirus), a parvovirus (e.g., parvovirus B19, canine parvovirus), a picornavirus (e.g., poliovirus, hepatitis A), a togavirus (e.g., rubella virus, alphaviruses such as chikungunya virus), a hepadnavirus (e.g., hepatitis B virus), a flavivirus (e.g., dengue virus, hepatitis C virus, West Nile virus, yellow fever virus, Zika virus, Japanese encephalitis virus), an orthomyxovirus (e.g., influenza A virus, influenza B virus, influenza C virus), a paramyxovirus (e.g., measles virus, mumps virus, respiratory syncytial virus, canine distemper virus, parainfluenza viruses), a rhabdovirus (e.g., rabies virus), a filovirus (e.g., Ebola virus), or a coronavirus or any other pathogenic virus discovered or yet to be discovered or combinations thereof.

In other embodiments, the pathogenic agent, biologic, therapeutic agent, or antigen (e.g., immunogenic agent) can be from a bacterium or a toxin of a bacterium. In certain embodiments, the pathogenic bacterium or toxin derived therefrom can include, but is not limited to, Pasteurella haemolytica, Clostridium difficile, Clostridium haemolyticum, Clostridium tetani, Corynebacterium diphtheria, Neorickettsia resticii, Streptococcus equi equi, Streptococcus pneumoniae, Salmonella spp., Chlamydia trachomatis, Bacillus anthracis, Yersinia spp., and Clostridium botulinum or any other pathogenic bacterium known or discovered or combinations thereof.

In some embodiments, the pathogenic agent, biologic, therapeutic agent, or antigen (e.g., immunogenic agent) can be from a fungus. In accordance with the embodiments, the fungus can include but is not limited to Cryptococcus spp. (e.g., neoformans and gatti), Aspergillus spp. (e.g., fumigatus), Blastomyces spp. (e.g., dermatitidis), Candida albicans, Paracoccidioides spp. (e.g., brasiliensis), Sporothrix spp. (e.g., schenkii and brasiliensis), Histoplasma capsulatum, Pneumocystis jirovecii and Coccidioides immitis, or any other pathogenic fungus known or discovered or combinations thereof.

In yet other embodiments, the pathogenic agent, biologic, therapeutic agent, or antigen (e.g., immunogenic agent) can be derived from a toxin. In accordance with these embodiments, the pathogenic agent, biologic, therapeutic agent, or antigen (e.g., immunogenic agent) derived from a toxin can include, but is not limited to, ricin toxin, botulinum toxin, Diphtheria toxin, tetrodotoxin, Pertussis toxin, snake venom toxins, and conotoxin or other known or discovered toxin or combinations thereof.

In some embodiments, PLP pathogenic agent, biologic, therapeutic agent, or antigen (e.g., immunogenic agent) or fragment thereof-containing particles and/or ALD coated particles disclosed herein can be used to manufacture one or more formulation or composition for use in any human, non-human mammal, animal, bird, or reptile (e.g., companion animal, pet or livestock). In accordance with these embodiments, formulations disclosed herein can be administered, for example, to a human of any age (e.g., fetus, infant, toddler, child, adolescent, young adult, adult or elderly adult), a dog (canine), a cat (feline), a horse (equine), a cow (bovine), a goat (hircine), a sheep (caprine), or a bird (e.g., poultry such as a chicken, turkey, duck, goose) or other mammal, animal or reptile to treat, reduce onset of or prevent a health condition.

In certain embodiments, PLP agent-containing or decorated PLP particles described herein can be used with or without ALD coating to generate one or more composition for administering to a canine to treat, reduce onset of or prevent an infection. In accordance with these embodiments, infections in a canine can include, but are not limited to, infections related to canine parvovirus (CPV), canine distemper virus (CDV), canine adenovirus (CAV), rabies, canine parainfluenza virus (CPiV), canine influenza virus, canine corona virus, measles virus, Bordetella bronchiseptica, Leptospira spp., and Borrelia burgdorferi or other infective organisms that infect a canine, or combinations thereof.

In some embodiments, PLP agent-containing or decorated PLP particles described herein can be used with or without ALD coating to generate one or more composition for administering to a feline to treat, reduce or prevent an infection an infection. In accordance with these embodiments, infections in a feline can include, but are not limited to, infections derived from or related to, feline herpesvirus 1 (FHV1), feline calicivirus (FCV), feline panleukopenia virus (FPV), rabies, feline leukemia virus (FeLV), feline immunodeficiency virus, virulent systemic feline calicivirus, Chlamydophila felis, Pasteurella haemolytica, and Bordetella bronchiseptica or other infective organisms that infect a feline, or combinations thereof.

In other embodiments, PLP agent-containing or decorated PLP particles described herein can be used with or without ALD coating to generate one or more composition for administering to an equine, to treat, reduce or prevent an infection. In accordance with these embodiments, an infection, can include, but is not limited to, infection derived from or related to, Eastern equine encephalomyelitis virus, Western equine encephalomyelitis virus, Venezuelan equine encephalomyelitis virus, bovine papillomavirus, rabies virus, Clostridium tetani, West Nile virus, equine influenza virus, Potomac fever (Neorickettsia risticii), Streptococcus equi equi, and rhinopneumonitis (equine herpesvirus type 1) or other infective organisms that infect an equine, or combinations thereof.

In certain embodiments, PLP agent-containing or decorated PLP particles described herein can be used with or without ALD coating to generate one or more composition for administering to a bovine, to treat, reduce or prevent an infection. In accordance with these embodiments, an infection, can include, but is not limited to, infection derived from or related to, bovine rhinotracheitis (IBR), parainfluenza type 3 (PI3), bovine virus diarrhea (BVD). bovine respiratory syncytial virus (BRSV), blackleg (Clostridium chauvoei), malignant edema (Clostridium septicum), infectious necrotic hepatitis (Clostridium novyi), enterotoxemia (Clostridium perfringens type C and D). Pasteurella haemolytica, and redwater (Clostridium haemolyticum) or other infective organisms that infect a bovine, or combinations thereof.

In some embodiments, PLP agent-containing or decorated PLP particles described herein can be used with or without ALD coating to generate one or more composition for administering to a human to treat. reduce onset of, prevent, or ameliorate a health condition. In certain embodiments. PLP agent-containing or decorated PLP particles with or without ALD coating described herein can be used to deliver one or more therapeutic agents to a human fetus, infant, child, adolescent, or adult including, but not limited to, therapeutic agents related to treat, reduce onset of, prevent, or ameliorate varicella-zoster (chicken pox), diphtheria, Haemophilus influenzae type b (Hib), hepatitis A, hepatitis B, influenza, measles, mumps, pertussis, polio, pneumococcal disease, rotavirus, rubella, and tetanus. In other embodiments, PLP agent-containing or decorated PLP particles with or without ALD coating described herein can be used to deliver one or more immunogenic compositions to a human fetus, infant, child, pre-teen or teen, adult or elderly adult, including but not limited to, therapeutic agents related to treat, reduce onset of, prevent, or ameliorate influenza, tetanus, diphtheria, pertussis, human papillomavirus, meningococcal disease, hepatitis B, hepatitis A, polio, measles, mumps, Severe acute respiratory syndrome (SARS), SARS coronavirus 2 (SARS-COV-2), RSV, rubella, and varicella-zoster or combinations thereof. In yet other embodiments, PLP agent-containing or decorated PLP particles with or without ALD coating described herein can be used to deliver one or more therapeutic agents to a human, including but not limited to, therapeutic agents related to treat, reduce onset of, prevent, or ameliorate influenza (e.g. A, B or C), tetanus, diphtheria, pertussis, zoster, pneumococcal disease, meningococcal disease, measles, mumps, rubella, varicella, hepatitis A, hepatitis B, Haemophilus influenzae type b or other pathogens or combinations thereof.

In other embodiments, PLP agent-containing or decorated PLP particles with or without ALD coating described herein can be used to generate compositions of use for administering to a human, including but not limited to, compositions against travel-related diseases or infections, including but not limited to, hepatitis A, hepatitis B, typhoid fever, paratyphoid fever, meningococcal disease, yellow fever, dengue fever, rabies, Zika virus infection, SARS-COV-2 infection. SARS infection, Ebola virus infection. Chikungunya disease, and Japanese encephalitis infection, or other travel-related conditions or combinations thereof.

In yet other embodiments, PLP agent-containing or decorated PLP particles with or without ALD coating described herein can be used to generate compositions of use for administering to a subject contemplated herein. In accordance with these embodiments, compositions can include, but are not limited to, therapeutic agent containing compositions against human papillomavirus (e.g. HPV 16, HPV18, HPV31. HPV45, or HPV 6 or HPV11.or any other HPV), herpes simplex virus, smallpox virus. rotavirus. parvovirus B19.chikungunya virus. dengue virus (e.g. dengue-1. dengue-2. dengue-3 or dengue-4). hepatitis C virus. West Nile virus, Zika virus, respiratory syncytial virus, rabies virus, and Ebola virus or other pathogenic microorganism or combination thereof.

In certain embodiments, PLP agent-containing or decorated PLP particles with or without ALD coating described herein can include a single agent dose or two or more doses of a particular agent or mixture of particles containing different therapies against different health conditions (e.g., prime and boost doses). In some embodiments, PLP agent-containing or decorated PLP particles with or without ALD coating described herein can include doses for two or more different pathogens.

In some embodiments, methods disclosed herein can concern controlled, ultra-rapid freezing rates combined with various concentrations of glass-forming or disaccharide agents. In accordance with these embodiments, these agents can include, but are not limited to, trehalose or sucrose or other disaccharides disclosed agent herein or capable of creating glassy matrices of use to stabilize compositions disclosed herein. In accordance with these embodiments, these agents can be used to generate glass-like matrices upon freezing to stabilize PLPs, PLP agent-containing or decorated PLP particles in an essentially dry form. One advantage of these matrices is that the PLPs, PLP agent-containing or decorated PLP particles are more tolerant to elevated temperatures (e.g., about 40° C. to about 70° C.) for extended periods of time. As disclosed herein, spray-drying buffer solutions containing disaccharides (glass forming agents) and polymeric carbohydrates (e.g., HES) increases the glass transition temperature for dried powder particles, conferring additional thermal stability to PLPs, and PLP agent-containing or decorated PLP. In certain embodiments, when the glass-forming agents are dried during a dehydration process (e.g., spray drying) in the presence of one or more PLP, PLP agent-containing or decorated PLP particles powders (glassy particles) are formed, containing embedded PLPs, PLP agent-containing or decorated PLP particles. In this dehydrated state, protein physical and chemical degradation pathways, which require molecular motion. can be inhibited, as are other degradation pathways thereby stabilizing the PLP microparticle associated agent or antigen or bioactive molecule (e.g., protein, polypeptide, polynucleotide, or the like). In accordance with these embodiments, particulates and/or essentially dry microparticles disclosed herein are formulated and spray dried such that therapeutic agents or phage of the undecorated or decorated phage or PLP formulations are not principally located at the surface of the spray dry particle (reduced exposure). It is known that high molecular weight compounds and low solubility compounds have a higher likelihood of ending up at the surface of a spray dried particle, therefore, embodiments disclosed herein address this commonly observed issue by specific additions to the formulation to form an external shell-like layer that is not principally the target therapeutic agent and can, for example, shield the polynucleotides making them more stable under certain conditions. In accordance with these embodiments, particulates and/or essentially dry microparticles disclosed herein can be introduced to an ALD reaction chamber where the particulates and/or essentially dry microparticles flow freely within the chamber for reduced agglomeration and/or aggregation of the particulates and/or essentially dry microparticles. It is noted herein that introduction of undecorated or decorated phage or PLPs directly to an ALD reaction chamber without creating thermostable particulates or essentially dry glassy microparticles as disclosed herein would be an unsuccessful coating protocol; for example, the elevated temperature would degrade the phage or PLPs or the phage or PLPs would likely stick to one another or the chamber and/or the metal material applied would not layer onto free PLPs or phage (decorated or undecorated; that are not encased in a protective shell or glassy particle) and/or fully encapsulate the decorated or undecorated phage or PLPs because there would be no availability of binding groups needed to secure the coating which is created herein using polymer-containing formulations to create the essentially dry microparticles or particulates disclosed herein for long term storage or for further coating. It was not known until the instant disclosure that formulations disclosed herein could create such a barrier or shell to protect the phage or PLPs disclosed herein from such conditions. It is understood by one of skill in the art that formulations and processes disclosed herein are scalable and readily available for manufacture for creating bulk microparticles for coating or storage and later coating.

In some embodiments, PLP agent-containing or decorated PLP particles with or without ALD coating disclosed herein can be produced with candidate antigens described herein. Candidate antigens can include but are not limited to antigens derived from or originating from or against: corona virus-related antigens, antigens of ricin toxin, Bacillus anthracis, Clostridium botulinum, human papilloma virus, Ebola virus, poliovirus, norovirus, rotavirus, hepatitis C, dengue virus derived antigens, varicella, herpes simplex, cytomegalovirus, Japanese encephalitis, West Nile virus, Zika virus and other related viruses such other flavivirus and alphavirus derived antigens known in the art. In some embodiments, bacterial antigens can be included in PLP agent-containing or decorated PLP particles with or without ALD coating disclosed herein, for example, antigens derived from or originating from or against Pneumococcus, Salmonella, and Clostridium difficile. In certain embodiments, the antigen can be a toxoid, for example, ricin toxoid, tetanus toxoid, diphtheria toxoid, and botulinum toxoid or other known or discovered toxoid. Other agents can be included in PLP agent-containing or decorated PLP particles with or without ALD coating disclosed herein, where the agent is capable of undergoing transitions and reconstitutions as described herein, including antigens of other bacteria, viruses, fungi, protozoans, or toxins.

In some embodiments, pathogenic agents and antigens derived therefrom contemplated herein can be in the form of a polynucleotide, of a recombinant peptide, a protein or other form, virus-like particles (VLPs), or inactivated or attenuated pathogens (e.g. viruses) or chimeric viruses or chimeric viruses in the same virus family such as flaviviruses. alphaviruses or the like. In certain embodiments, human papilloma virus (HPV) capsomeres can be incorporated into PLP agent-containing or decorated PLP particles disclosed herein via spray-drying without effect on the morphology of the HPV capsomeres. In certain embodiments, capsomeres from two or more HPV can be incorporated into the same PLP agent-containing or decorated PLP particles. For example, in some embodiments, capsomeres from HPV types 16, 18, and 31 can be incorporated into the same PLP agent-containing or decorated PLP particles to produce a trivalent particle. In other embodiments, capsomeres from HPV types 16, 18, 31, and 45 can be incorporated into the same PLP agent-containing or decorated PLP particles to produce a tetravalent particle. In certain embodiments, the capsomere from each HPV type is the L1 protein. Many different combinations of capsomeres from different HPV types can be incorporated PLP agent-containing or decorated PLP particles disclosed herein. Recombinant peptides, polynucleotides, or protein immunogens from pathogens other than HPV can be incorporated into PLP agent-containing or decorated PLP particles disclosed herein, similarly to L1 HPV capsomeres.

In yet other embodiments, inactivated or attenuated pathogens (e.g. live, attenuated viruses) can decorate phage or PLPs disclosed herein and spray-dried into thermostable particles. In accordance with these embodiments, inactivated (or killed) viruses or virus particles, bacteria, or other pathogens can be inactivated by any means, for example, chemically or by heat and incorporated into these particles. Non-limiting examples of inactivated pathogens that can decorate PLP particles can include inactivated whole-cell pertussis (inactivated Bordetella pertussis), Salmonella typhi, and inactivated polio virus. Live, attenuated viruses or bacteria can similarly decorate PLP particles. Non-limiting examples of attenuated viruses and bacteria that can incorporate PLP particles and can include measles virus, mumps virus, rubella virus, influenza virus, chicken pox virus, smallpox virus, polio virus, rotavirus, flaviviruses (e.g. dengue virus, yellow fever virus), rabies virus, typhoid virus, Mycobacterium bovis, Salmonella typhi, and Rickettsia spp.

In some embodiments, aluminum salts of use to supplement compositions disclosed herein to induce immune reactivity can include one or more of aluminum hydroxide. aluminum phosphate and aluminum sulfate, or combinations thereof. In accordance with these embodiments. the aluminum salt can be in the form of an aluminum hydroxide gel (e.g., Alhydrogel®). In some embodiments, the decorated and coated PLPs disclosed herein can induce enhanced immune responses without the need for additional adjuvants, for example, as alumina sparing.

In some embodiments. buffers of use herein prior to spray-drying PLP decorated or undecorated particles can include, but are not limited to, histidine, glycine, arginine, phosphate, citrate, acetate, Tris(Hydroxymethyl)aminomethane, succinate, prolamine, borate, carbonate or a combination thereof. In some embodiments, the buffer of use herein prior to spray-drying includes, but is not limited to, histidine, glycine, arginine, phosphate, citrate, acetate, Tris(Hydroxymethyl)aminomethane, or a combination thereof. In certain embodiments, a buffer can further include, but is not limited to, acetate, succinate, citrate, histidine, glycine, prolamine, borate, phosphate, formate, carbonate, and the like or a combination thereof. In other embodiments, a buffer can further include at least one volatile salt including, but is not limited to, one or more of sodium succinate, potassium succinate, sodium phosphate, potassium phosphate, ammonium acetate, ammonium formate, ammonium carbonate, ammonium bicarbonate. triethylammonium acetate, triethylammonium formate, triethylammonium carbonate, trimethylamine acetate trimethylamine formate, trimethylamine carbonate, pyridinal acetate and pyridinal formate, and the like, or combinations thereof. In certain embodiments, the buffer can include histidine, for example, histidine-HCl. In certain embodiments, the volatile salt can include ammonium acetate. In yet other embodiments, one or more salts can be included in the compositions prior to spray-drying (e.g., magnesium sulfate, MgSO₄)

In some embodiments, glass-forming or disaccharide agents of use herein can include one or more of trehalose, sucrose, ficoll, dextran, sucrose, maltotriose, lactose, mannitol and glycine, glycine, cyclodextrin, and povidone, or combinations thereof. In certain embodiments, the glass-forming agent can be trehalose. In other embodiment, the trehalose concentration can be present in a weight-to-volume (w/v) concentration from about 0.1% to about 40% in an immunogenic composition prior to dehydration: from about 1% to about 30% w/v; from about 5% to about 20%; or from about 8% to about 15% w/v in the immunogenic composition prior to dehydration. In another embodiment, the glass-forming agent can be trehalose in a concentration from about 8% to about 11%: or about 9.5% w/v in the immunogenic composition prior to dehydration.

In certain embodiments, a smoothing excipient of use in compositions and methods disclosed herein can be included in the composition to be lyophilized. In accordance with embodiments disclosed herein, the smoothing excipient can aide in creation of a smooth(er) particle surfaces, which in turn can create improved ability to deposit one or more coating layers on the particles. In accordance with these embodiments, having a smooth (er) particle with reduced inconsistencies on the surface can reduce the risk of cracking. In other embodiments, a rough surface may be desired to aid in response to an administered particle to a subject (e.g., increase immune responses) In certain embodiments, coating layers described herein can be about 0.1 nm to about 30.0 nm in thickness. In certain embodiments, the smoothing excipient can also function as a glass-forming agent. In some embodiments, the smoothing excipient can be hydroxyethyl starch or another pharmacologically acceptable plasma expander including, but not limited to, serum albumin, human serum albumin, dextran, hetastarch, and plasma protein factor, or the like or a combination thereof.

In other embodiments, the smoothing excipient can be hydroxyethyl starch. In some embodiments, the smoothing excipient can be present in a weight-to-volume (w/v) concentration from about 0.1% to about 40% in a composition prior to spray-drying or lyophilizing. In other embodiments, the smoothing excipient is different from the glass-forming agent or disaccharide agent, and its concentration is from about 0.1% to about 20%. In some embodiments when the smoothing excipient is different from the glass-forming agent, the smoothing excipient concentration is about 0.1% to about 5%: about 0.1% to about 2.5%: about 0.1% to about 1.0%, about 0.1% to about 0.5%, or about 0.1% to about 0.25% in a composition prior to spray-drying. In other embodiments, where the smoothing excipient also functions as the glass-forming agent, the smoothing excipient can be present in a weight-to-volume (w/v) concentration from about 0.1% to about 40% in a composition prior to spray-drying. In some embodiments where the smoothing excipient functions as the glass-forming agent, the smoothing excipient can be in a concentration of about 1.0% to about 30% w/v, about 5.0% to about 20% (w/v). or about 8% to about 15% (w/v) in a composition prior to spray-drying or dehydration.

In some embodiments, formulations disclosed herein can further include at least one surfactant. In accordance with these embodiments, formulations of use to spray dry PLP or loaded/decorated PLP formulations can include at least one surfactant. In some embodiments, compositions disclosed herein can be enhanced by the addition of nonionic surfactants. In accordance with these embodiments, surfactants can be added to formulations before or after spray drying or dehydration at concentrations ranging from approximately 0.1 times the critical micelle concentration of the surfactant in the formulation. to approximately 20 times the critical micelle concentration of the surfactant in the formulation before, during or after spray drying of the composition. Suitable nonionic surfactants include, but are not limited to, polsorbates such as Tween 20. Tween 40. Tween 60 and Tween 80. poloxamers for example Poloxamer 188 and Poloxamer 407, Poloxamer 235, Poloxamer 335, Brij, alkylphenol hydroxypolyethylene surfactants such as Triton X100. Triton X114 and Triton X405, and Oligoethylene glycol monoalkyl ethers such as Genapol or the like or other suitable surfactant.

In some embodiments, formulations disclosed herein can include decorated or undecorated phage or PLPs, at least one disaccharide, at least one smoothing agent, at least one surfactant. at least one volatile salt and a buffer. In accordance with these embodiments, a formulation can include where the at least one disaccharide includes trehalose. the at least one smoothing agent includes HES, the at least one surfactant includes Tween80, the at least one volatile salt includes ammonium acetate and the at least one buffer includes histidine.

In certain embodiments, agents used to decorate phage or PLPs (ALD coated or uncoated) of the present disclosure can be of use for prophylactic and/or therapeutic immunogenic compositions. Suitability of agents for use in phage or PLP agent-containing particles can be tested by reaction with antibodies or monoclonal antibodies which react or recognize conformational epitopes present on the intact target of the decorating agent and based on the agent's ability to elicit the production of neutralizing antiserum. Suitable assays for determining whether neutralizing antibodies are produced are known to those of skill in the art. In this manner, in certain embodiments, it can be verified whether the agents of the present disclosure will elicit production of neutralizing antibodies.

In certain embodiments, phage, PLPs or decorated phage or decorated PLPs stabilized as glassy particles or essentially dry particles post spray draying or other form of dehydration can be coated by ALD or sequestered in one or more of the ALD coating layers of coated glassy particles contemplated herein. Certain embodiments concern using molecular deposition processes such as ALD coating, in methods, compositions, and formulations for generating single-administration, multi-dose, thermostable phage or PLP-containing decorated or undecorated compositions. In some embodiments, decorated or undecorated PLP thermostable particles can be generated and coated by ALD where the coating or sequestering layers not only serve as adjuvants to induce an immune response, but also provide for temporally-separated first, second, third or more doses in a single particle for single administration to a subject (e.g., prime and boost for example of an immunogenic agent containing PLP or phage). In some embodiments, PLP or phage thermostabilization can be achieved by a combination of embedding one or more PLP or phage into glassy organic matrices to form one or more phage- or PLP-containing glassy particles (e.g., decorated or undecorated). and by using molecular deposition processes that enable a wide variety or range of the same or different molecular layers (e.g., aluminum, titanium or other organic metal) to be applied to the one or more phage- or PLP-containing glassy particles and encasing the one or more phage- or PLP-containing glassy particles to obtain encased or coated phage- or PLP-containing glassy particles. In accordance with these embodiments, the thickness of these coating or sequestering layers can be controlled to within 1 or 2 angstroms in thickness and can range from about 0.1 nm to about 30.0 nm or about 0.1 nm to about 20.0 nm per layer. In accordance with these embodiments, one or more coating layers can be deposited consecutively upon one another in a single coating event within an ALD reactor, for example. In certain embodiments. the thickness of these coating or sequestering layers can be from about 5.0 nm to about 25.0 nm. Using a series of alternating, self-limiting surface reactions, these coating layer deposition processes are highly scalable. For example, fluidized bed reactors can be used to allow large bulk quantities of phage- or PLP-containing glassy particles (e.g., decorated, or undecorated with an agent) to be coated with coating layers without agglomeration of the particles. This process allows for complete encasement of the one or more phage- or PLP-containing glassy particles and can protect the one or more phage- or PLP-containing glassy particles from immediate deposition when administered to a subject.

In some embodiments, molecular deposition techniques such as ALD can be used to apply nanometer-thick coatings of metalloorganic such as metal oxide or metal alkoxide, or other metal containing agent onto a surface of phage- or PLP-containing glassy particles (e.g., decorated, or undecorated with an agent). In certain embodiments, the coating or sequestering layer can be an aluminum-based material including, for example, an aluminum oxide or an aluminum alkoxide (e.g., alucone), or a material of silicon dioxide (SiO₂). titanium dioxide (TiO₂), zinc oxide (ZnO₂) or silicon nitride (Si₃N₄) alone or in a suitable combination composition or alternating or other suitable layering combination thereof. In accordance with these embodiments, the metal-containing material is deposited on or applied to the surface of the one or more phage- or PLP-containing glassy particles (e.g., decorated, or undecorated with an agent) to coat or sequester the one or more phage- or PLP-containing glassy particles (e.g., decorated. or undecorated with an agent) in 1, 2, 3, 4, 5, 6, 7, 8, 9, 10, 11, up to 20, up to 30, up to 40, up to 50, up to 100, up to 150, up to 200, up to 250 or more layers of the metal-containing material to form encased phage- or PLP-containing glassy particles (e.g., decorated, or undecorated with at least one agent). In certain embodiments, the metal-containing material is deposited on or applied to the surface of the one or more phage- or PLP-containing glassy particles (e.g., decorated, or undecorated with an agent) to coat or sequester the one or more phage- or PLP-containing glassy particles (e.g., decorated, or undecorated with an agent) in at least 10, at least 20, at least 30 or at least 40 coating layers of the metal-containing material to form encased phage- or PLP-containing glassy particles (e.g., decorated, or undecorated with at least one agent).

In accordance with these embodiments, some advantages of depositing one or more ALD applied coating layers on phage- or PLP-containing glassy particles (e.g., decorated with at least one agent, or undecorated) include, but are not limited to, the coating layers can dissolve slowly or at a pre-determined rate when the particles are administered to a subject, thus allowing temporal control of the release of the particle contents (e.g., the one or more decorating agents). Release times can be tailored by adjusting composition(s) of the coating layers and number and/or thickness of molecular layers applied to the microparticles. In some embodiments, about 5 to about 100 or about 5 to about 200, or about 10 to about 300, or about 10 to about 500 layers or more coating or sequestering layers can be used to form the coated or sequestered phage- or PLP-containing glassy particles (e.g., decorated with at least one agent, or undecorated) of the present disclosure. In some embodiments, release of the agents from the coated or sequestered particle's core can occur within hours, to about 1 day, or about 7 days or about 30 days or about 60 days or about 90 days or about 120 days or about 180 days or any timing in between after administration to the subject. In other embodiments, release of the one or more innermost sequestered or coated PLP-containing agents from the microparticle can occur from about 10 days to about 90 days after administration to the subject. In some embodiments, release of the innermost agent can occur from about 30 days to about one year, or about 30 days to about 150 days, or about 30 days to about 120 days, or about 30 days to about 60) days, after administration to the subject. In some embodiments, release of an innermost agent of the phage- or PLP-containing glassy particles (e.g., decorated with at least one agent, or undecorated) can occur from about 10 days to about 320 days after administration to the subject. In some embodiments, release of the innermost agents can occur from about 10 days to about 180 days after administration to the subject. Further, in some embodiments. release of the innermost agents can occur from about 10 days to about 120 days after administration to the subject.

In certain embodiments, particle size of the uncoated or encased phage- or PLP-containing glassy particles (e.g., decorated with at least one agent or undecorated) is from about 10 nm to about 90 nm, or about 20 nm to about 80 nm. In other embodiments, encased phage- or PLP-containing glassy particles having multiple layers is less than about 5 μm in size. It will be recognized that the elements of the phage- or PLP-containing glassy particles, coating layers, and any additional layers can be provided in concentrations capable of providing a suitable dose of one or more target agent (e.g., decoration) while maintaining an appropriate particle size for ease of reconstitution and delivery, for example, It is noted that the coatings disclosed herein include nanometer-sized layers and do not effectively change the size of any of the essentially dry microparticles being coated, even with an application of a large number of coatings (e.g., up to 300 or more). In certain embodiments disclosed herein, decorated PLPs can be from about 1.0% up to 100% (e.g., about 10%) decorated to be effective, depending on the health condition targeted, the phage of interest and the decoration(s) to be applied to the phage.

In certain embodiments, one advantage of using one or more aluminum-based materials as coating or sequestering layer(s) and/or titanium-based materials is that the aluminum-based materials and/or titanium-based materials can also act as an adjuvant. In accordance with these embodiments, the aluminum-based and/or titanium-based materials (e.g., aluminum oxide, aluminum alkoxide and/or titanium oxide or the like) coating layers sequestering or surrounding the phage- or PLP-containing glassy particles (e.g., decorated with at least one agent or undecorated) expose essentially the same surface chemistries to immunoactive cells as do standard aluminum-based or other adjuvant agents known in the art. In some embodiments, nanoscopic aluminum-based coating layers layered on phage- or PLP-containing glassy particles (e.g., decorated with at least one agent or undecorated) disclosed herein can be significantly thinner than what is found in particles of conventional vaccines; therefore, total amounts of aluminum per administration will be essentially negligible, enhancing safety and reduced side effects of these agents. In certain embodiments, the aluminum-based coating layer can be sufficiently thin so that the total aluminum concentration per administration of the immunogenic composition to a subject is less than about 100 μg, less than about 20 μg, less than about 10 μg, less than about 5 μg, or less than about I ug or even less.

In some embodiments, coating layers other than aluminum-based coating layers can be used in order to coat or sequester the phage- or PLP-containing glassy particles (e.g., decorated with at least one agent or undecorated). In accordance with these embodiments, non-aluminum coating layers including, but not limited to, silicon dioxide (SiO₂), titanium dioxide (TiO₂), zinc oxide (ZnO2) or silicon nitride (Si₃N₄) can be used either in combination with aluminum-based coating layers, or alone to the exclusion of aluminum-based coating layers. With each type of material having different characteristic dissolution times, layers of different materials can be deposited on the particles to vary the temporal release of the agent (e.g., decoration) from the particles core or outer coating layer as appropriate. In some embodiments, particles can be coated with one or more aluminum-based layers. followed by one or more layers of a different material such as alternating materials or some other pattern, as desired. In accordance with these embodiments, different materials can dissolve more slowly than aluminum-based coating layer to accurate timed release of a target agent from the phage or PLPs. In other embodiments, using other materials for coating the particles, can reduce the number of aluminum-based layers necessary to provide for a given release time, minimizing the amount of aluminum per dose.

In some embodiments, one or more coating layers can be deposited on a phage- or PLP-containing glassy particle (e.g., decorated with at least one agent or undecorated) by, for example, atomic layer deposition (ALD, for example any instrumentation capable of atomic layer deposition can be used). ALD includes a thin film deposition technique that is based on the sequential use of a gas phase chemical process. ALD is considered a type of chemical vapor deposition. In certain methods, most ALD reactions use two chemicals, referred to as precursors. In accordance with these embodiments, these precursors can react with the surface of a material one at a time in a sequential, self-limiting, or directed manner. Through the repeated exposure to separate precursors, a thin film can be slowly deposited. Use of ALD to deposit coating layers on phage- or PLP-containing glassy particles (e.g., decorated with at least one agent or undecorated) can be based on sequential, self-limiting reactions and provides for layer thickness control at the Angstrom level and tunable coating layer composition.

In certain embodiment, the ALD method can be optimized for a particular situation or condition. For example, antigens against a pathogenic organism decorating phage or PLPs can have variable thermostability, and therefore might not be amenable to higher ALD temperatures due to this vulnerability. In certain embodiments, molecular deposition can occur at temperatures at which the at least one PLP decoration of a PLP agent-containing glassy particle remains stable. In some embodiments, molecular deposition can occur under vacuum conditions. By performing the molecular deposition under vacuum conditions, coating layers can be applied at reduced temperatures and reduce or eliminate adverse effects of higher temperatures on the phage- or PLP-containing glassy particle (e.g., decorated with at least one agent or undecorated). In certain embodiments, vacuum conditions required for deposition can be minimal. In some embodiments, the ALD process can occur under a mild vacuum of about 0.1 atmospheres. In other embodiments, ALD can also include incorporation of magnetically-coupled powder mixing devices that can lead to shorter cycle times for deposition of the material by providing uniform distribution of powders and reactants within an ALD reactor. In accordance with these embodiments, once a phage- or PLP-containing glassy particle (e.g., decorated with at least one agent or undecorated) is introduced to an ALD device for coating. it remains within the ALD device until completion of the desired coating layers. For example, a phage- or PLP-containing glassy particle (e.g., decorated with at least one agent or undecorated) can be introduced to an ALD coating device (e.g., ALD reaction chamber) and up to 10, 20, 30, 40, 50, 100, 150, 200, 250 or more coating layers can be applied to completion for the desired number of coating layers.

Embodiments of the present disclosure, provide for thermostable phage- or PLP-containing glassy particle (e.g., decorated with at least one agent or undecorated) uncoated or coated compositions. where the thermostable composition can be produced by formulating uncoated or coated phage- or PLP-containing glassy particle (e.g., decorated with at least one agent or undecorated) into a composition. In certain embodiments, these compositions can be used as vaccines or other therapeutics to treat, reduce onset of, or prevent a health condition.

In some embodiments, as described herein, thermostable phage- or PLP-containing glassy particle (e.g., decorated with at least one agent or undecorated) coated compositions reduce incompatibilities between two or more different agents, whether during storage or after being administered to a subject. In other embodiments, phage- or PLP-containing glassy particle (e.g., decorated with at least one agent or undecorated) uncoated or coated compositions can include two or more different agents (e.g., two or more different antigens) in the same coated or uncoated phage- or PLP-particle. In these embodiments, the phage- or PLP-containing glassy particle (e.g., decorated with at least one agent or undecorated) are safeguarded against incompatibilities between the two or more different agents due to their stabilization within a glassy matrix (e.g., reduced molecular movement etc.), and in certain embodiments due to physical separation of the two or more agents by one or more ALD coating layers.

Certain embodiments of the present disclosure include methods to elicit an immune response to an agent decorating a phage- or PLP-containing glassy particle (uncoated or coated) alone or in combination with other agents by administering to the subject a composition including phage- or PLP-containing glassy particle (e.g., decorated with at least one agent) disclosed herein. The composition including the phage- or PLP-containing glassy particle (e.g., decorated with at least one agent) can be administered in therapeutically effective amounts. That is, in amounts sufficient to produce a protective immunological response. Generally, these compositions can be administered in active agent dosages ranging from about 0.001 mg to about 20.0 mg agent or about 0.01 mg to about 20.0 mg agent, or about 0.1 mg to about 10.0mg agent. Single or multiple dosages can be administered in a single administration composition. Where multiple doses of an immunogenic or vaccine composition are administered in a single administration composition, for example, in prime-boost compositions, one of the two doses can be temporally controlled for release at a pre-selected time after administration.

In certain embodiments, administration of compositions disclosed herein can be performed using any acceptable means (e.g., parenterally, locally, or systemically, including by way of example, intravenously, orally, intranasally, subcutaneously, intradermally, by aerosol, intravaginally, by suppository, intramuscularly, intraocularly, and topically and other suitable administration). In some embodiments, administration of formulations or compositions disclosed herein can be affected by factors including a natural route of infection by a particular pathogen or location of a health condition within a subject's body such as an organ, blood, lymph nodes or other location. Dosage or dosages administered can depend upon factors including the age, health, weight, kind of concurrent treatment, exposure, if any, and the nature and type of the particular agent. Compositions or formulations containing one or more phage- or PLP-containing glassy particle (e.g., decorated with at least one agent or undecorated) coated or uncoated disclosed herein can be employed in multi-dose form in a single vial or in dosage form such as capsules, liquid solutions, suspensions, or elixirs, for oral administration, or sterile liquid formulations such as solutions or suspensions for parenteral or intranasal use. Kits

Other embodiments provide kits for creating phage- or PLP-containing glassy particle (e.g., decorated with at least one agent or undecorated) coated or uncoated disclosed herein. In other embodiments, kits are contemplated of use for compositions, and methods described herein. Kits can be portable. In certain embodiments, kits can be used to transport to and be used in remote areas such as military installations or remote villages. The thermostability of the PLP agent-containing glassy microparticles allows for transport and storage without the need for a cold chain (e.g., refrigeration).

In other embodiments, kits can include an appropriate carrier or diluent suitable for reconstituting the thermostable or essentially dry phage- or PLP-containing glassy particle (e.g., decorated with at least one agent or undecorated) coated or undecorated formulations. In certain embodiments, the carrier or diluent can be a pharmaceutically acceptable aqueous buffer suitable for injection that includes pyrogen-free water and can resist changes in pH upon addition of an inorganic compound, organic compound, acid, alkali, or dilution with a solvent or diluent.

In some embodiments, kits can include one or more suitable containers, for example, vials, tubes, mini- or microfuge tubes, test tube, flask, bottle, syringe, or other container. Where an additional component or agent is provided, the kit can contain one or more additional containers into which this agent or component may be placed. Kits herein will also typically include a means for containing the immunogenic agent-containing agents and/or immunogenic agent-containing glassy microparticles, pharmaceutically acceptable carrier or diluent and any other reagent containers in close confinement for commercial sale. Such containers may include injection or blow-molded plastic containers into which the desired vials are retained. Kits disclosed herein can further include instructions for making phage- or PLP-containing glassy particle (e.g., decorated with at least one agent or undecorated) coated or uncoated compositions or for administering phage- or PLP-containing glassy particle (e.g., decorated with at least one agent or undecorated) coated or uncoated to a subject.

### EXAMPLES

The materials, methods, and embodiments described herein are further defined in the following Examples. Certain embodiments are defined in the Examples herein. It should be understood that these Examples, while indicating certain embodiments, are given by way of illustration only. From the disclosure herein and these Examples, one skilled in the art can ascertain the essential characteristics of this invention, and without departing from the spirit and scope thereof, can make various changes and modifications of the invention to adapt it to various usages and conditions.

Atomic layer deposition (ALD) can deposit nanoscopic layers of alumina on spray-dried particulate agents (e.g., vaccines). Using these techniques, particles can be coated in a fluidized bed reactor by alternating injections of gas-phase reagents (in the present case, trimethylaluminum and water) to deposit single molecular layers of alumina in self-limiting reactions. Multiple cycles of alternating injections can be used to grow uniform alumina layers of arbitrary, controlled thickness on particle surfaces. When agent-containing powders coated with these nanoscopic metal oxide or metal alkoxide layers are injected, the layers dissolve slowly, providing controlled release of antigens encapsulated within the cores of coated particles and allowing both prime and boost doses of an agent to be administered in a single shot. Furthermore, the nanoscopic coatings can also serve as adjuvants.

It was hypothesized that combining tunable PLP systems with spray drying and ALD coating could result in a platform with the potential to produce rapidly adaptable vaccines that would be thermally stable and deliverable in a single shot administration. To evaluate this possibility, stability of liquid suspensions of phage were first compared against spray dried formulations by measuring phage infectivity by plaque assays after incubation for one year at room temperature or 37° C. Next, phage-derived PLPs were decorated with a model antigen. These decorated PLPs were spray dried to form glassy particles, coated with nanoscopic layers of metal oxide or metal alkoxide in a fluidized bed ALD reactor, and incubated for one month at 4° C. or 50° C. for stability analysis and other features. Physical stability of PLPs were measured. Then, doses of alum-adjuvanted liquid and reconstituted spray dried formulations or single doses of ALD-coated formulations were administered to mice by intramuscular injection, and immune responses were analyzed.

### Example 1

### Formulated Phage λ

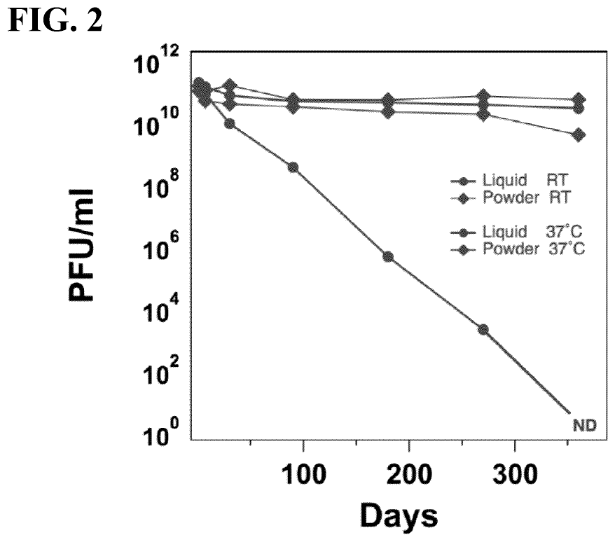

In one exemplary method, phage platform systems for delivering timed-release therapeutic agents were investigated. Phages are typically stored in buffered solutions and are stable for extended periods of time at 4° C. However, maintaining cold chain temperatures at 4° C. poses logistical problems for widespread therapeutic agent distribution. Therefore, stabilization of an infectious bacteriophage by spray drying were investigated. For this example, purified phage λ was spray dried and the resulting powders were stored at room temperature or 37° C. for pre-determined periods. Then these spray dried powders were reconstituted in water at the indicated times and evaluated for infectivity via plaque assays, comparing the powder samples to liquid controls that had been stored at room temperature or 37° C. (see for example, FIG. 2). As projected, liquid samples were stable when stored at room temperature; however, a significant loss of infectivity was observed when the liquid control samples were stored at 37° C., where the infectious titers dropped by approximately 8 orders of magnitude over nine months of storage and by one year there was no detectable titer in these liquic control samples. In contrast, the formulated preparations (e.g., with glass forming agents) that were spray dried (red diamonds) were stable at the temperatures tested, even at 37° C. for one year with very little loss.

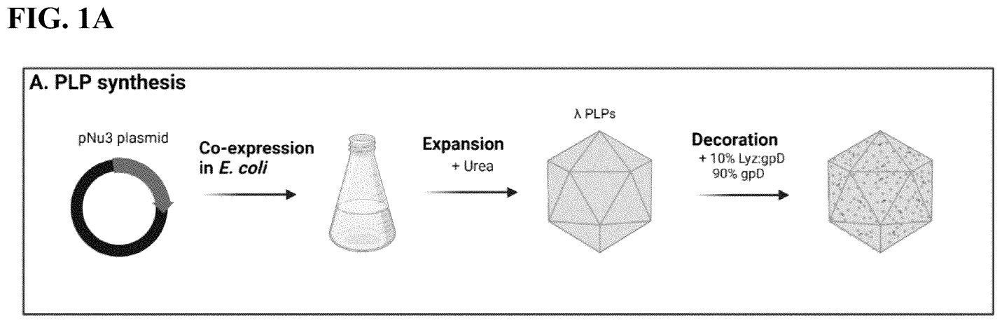

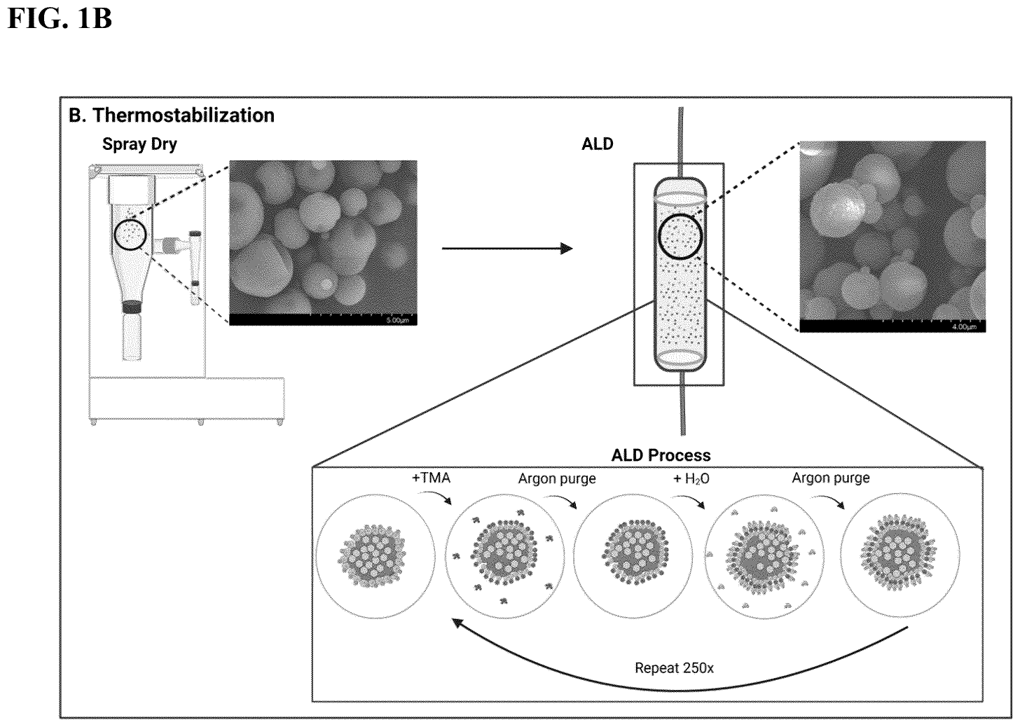

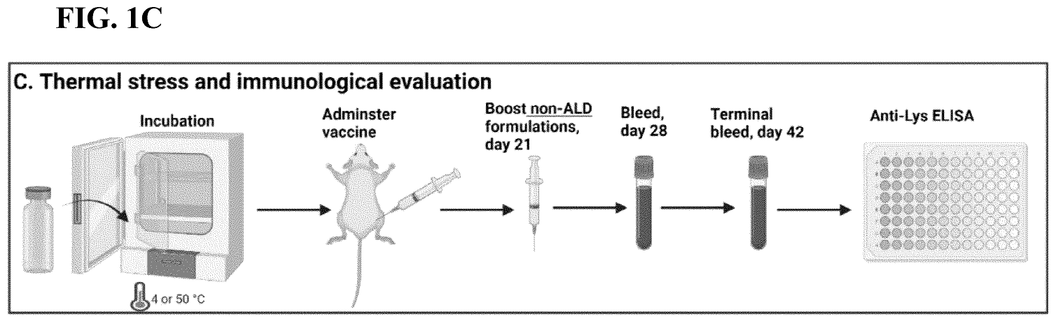

FIG. 1. Illustrates on study overview in a schematic of certain examples disclosed herein. 1A represents creation of λ PLPs through expression of a pNu3 plasmid, which forms a λ procapsid. After expansion with urea, gpD:T4-lysozyme is added and subsequently decorated on the shell created by these processes. IB represents decorated PLPs (Lys-PLPs) suspended in a buffered solution containing trehalose-hydroxyethyl starch and then spray-dried. Spray-drying buffer solutions containing disaccharides (glass forming agents) and polymeric carbohydrates increases the glass transition temperature for dried powder particles, conferring additional thermal stability to the Lys-PLPs. Then, the spray dried particles are coated using atomic layer deposition (ALD), a process that alternates for example, doses of trimethylaluminum (TMA) and water between argon purges. Each cycle of TMA deposits an approximately 2.3 Å-thick layer of metal material (e.g., alumina) on the surface of each thermostable decorated PLP particle. The alumina acts not only as an adjuvant but also prevents intrusion of water inside the coating, further stabilizing the particles for long term storage and for timed release of the decorations/therapeutic agents carried by the PLPs. 1C illustrates a schematic of after incubation at 4° C. or 50° C. for about one-month, liquid formulations of decorated PLPs (Lys-PLPs), spray dried Lys-PLP powders, and ALD coated particles were injected in 5 μg doses in BALC/C mice. Booster doses of all formulations except the ALD formulations were administered 21 days after the initial prime dose. Blood was collected on days 0, 28, and 42 after the initial injection.

FIG. 2 illustrates stability of formulated spray dried phage. Phage λ was incubated at room temperature (green plotted lines) or 37° C. (red plotted lines) as either liquid (circles) or spray dried powders (diamond) formulations. Phage λ maintained infectivity in all formulations and at all incubation temperatures tested except for the liquid formulation incubated at 3720 C. which by one year had no detectable titer (depicted as ND on the graph).

### Example 2

### Vaccine Biophysical Characterization

In another exemplary method, the decorated PLPs (e.g., Lys-PLPs) in liquid, spray dried (SD), and ALD coated formulations after 4° C. and 50° C. incubations were analyzed to determine if the antigen had been damaged due to thermal stress. Spray dried formulations were reconstituted to their original volumes by addition of purified water. Reconstitution was complete within a few minutes. ALD coatings applied to the spray dried powders rendered them insoluble in water: to analyze the PLPs within the spray dried powder cores the coating layer was first removed as described above using a sodium phosphate buffer solution.

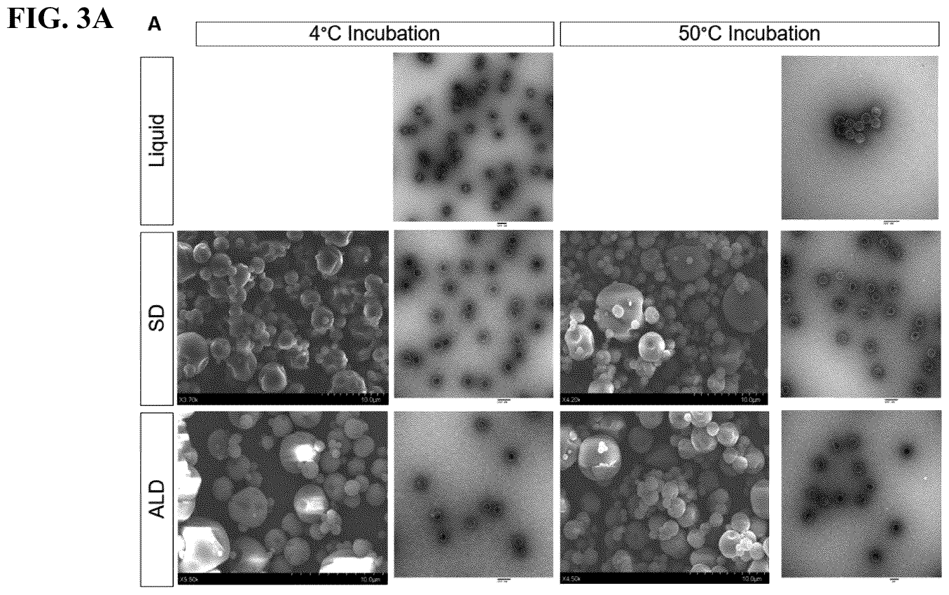

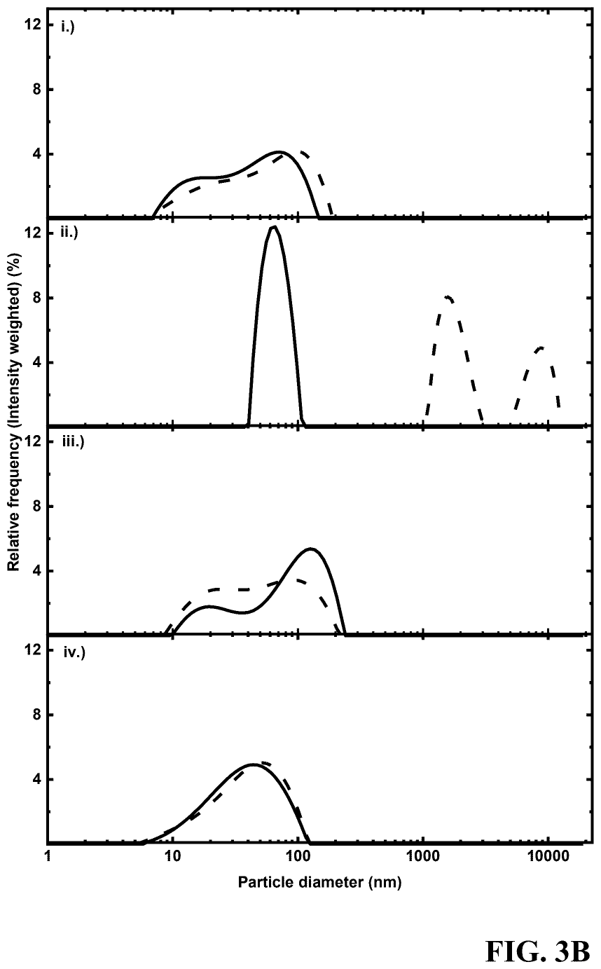

In another exemplary method, size and morphology of decorated PLPs (Lys-PLPs) were analyzed by transmission electron microscopy (TEM), scanning electron microscopy (SEM) (FIG. 3A) and dynamic light scattering (DLS) (FIG. 3B). The TEM micrographs and DLS results demonstrated that the decorated PLPs (Lys-PLPs) size is around 70 nm. slightly larger than the 60-nm wild-type PLP as a result of surface decoration. No apparent physical damage to the PLPs in the spray dried and ALD formulations was visible by TEM after incubation as the overall PLP morphology remained unchanged, and aggregation of the PLPs was not evident. In the liquid formulations, however, the PLPs appeared to be aggregated during incubation at 50° C. as indicated by both TEM and DLS.SEM analysis after incubation of spray dried powders demonstrated no melted particles nor particle necking (e.g., necking is when particles partially fuse together) which would have indicated the particles had absorbed water during the incubation.

Size distributions of the PLPs were analyzed using DLS (FIG. 3B) before spray drying and after reconstitution of the SD powder (FIG. 3B. I. top panel) to confirm the PLPs remained undamaged in the buffer and after the spray drying process prior to incubation. A peak in the size distribution at approximately 20 nm was observed in all SD and ALD formulations containing hydroxyethyl starch. No changes in the size distribution of PLPs in the liquid formulation were observed after incubation at 4° C. (FIG. 3B. ii. second panel from top), whereas size distribution changes in the liquid formulation incubated at 50° C. indicated that severe aggregation of PLPs had occurred. The SD and ALD formulations that were incubated at 4° C. or 50° C. did not exhibit substantial size distribution changes (FIG. 3B iii (third panel from top) and iv, bottom panel). These observations suggest that SD and ALD particles maintained their integrity during storage at temperatures up to 50° C. and were thermostable compared to liquid formulations.

FIGS. 3A-3B represents images related to biophysical characterization of spray dried and ALD-coated Lys-PLPs after incubation at 4° C. or 50° C. 3A.) illustrates SEM images of spray dried particles (middle row, left columns) or ALD-coated particles (bottom row, left columns) incubated at 4° C. (left) or 50° C. (right). TEM images of liquid Lys-PLPs incubated at 4° C. (left) or 50° C. (right) resuspended spray dried particles (middle row) or ALD-coated particles (bottom row) incubated at 4° C. (left) or 50° C. (right). 3B.) illustrates size distributions determined by DLS for Lys-PLPs in liquid suspensions prior to spray drying (solid line. i.) and in reconstituted spray dried powder after spray drying (dashed line. i.). Liquid Lys-PLP formulation incubated at 4° C. (solid line, ii) or 50° C. (dashed line. ii). Spray dried Lys-PLPs formulation incubated at 4° C. (solid line. iii) or 50° C. (dashed line, iii). ALD-coated Lys-PLP formulations incubated at 4° C. (solid line, iv) or 50° C. (dashed line, iv).

### Example 3

### Immunogenicity of Lys-PLPs Vaccine Formulations After Incubation

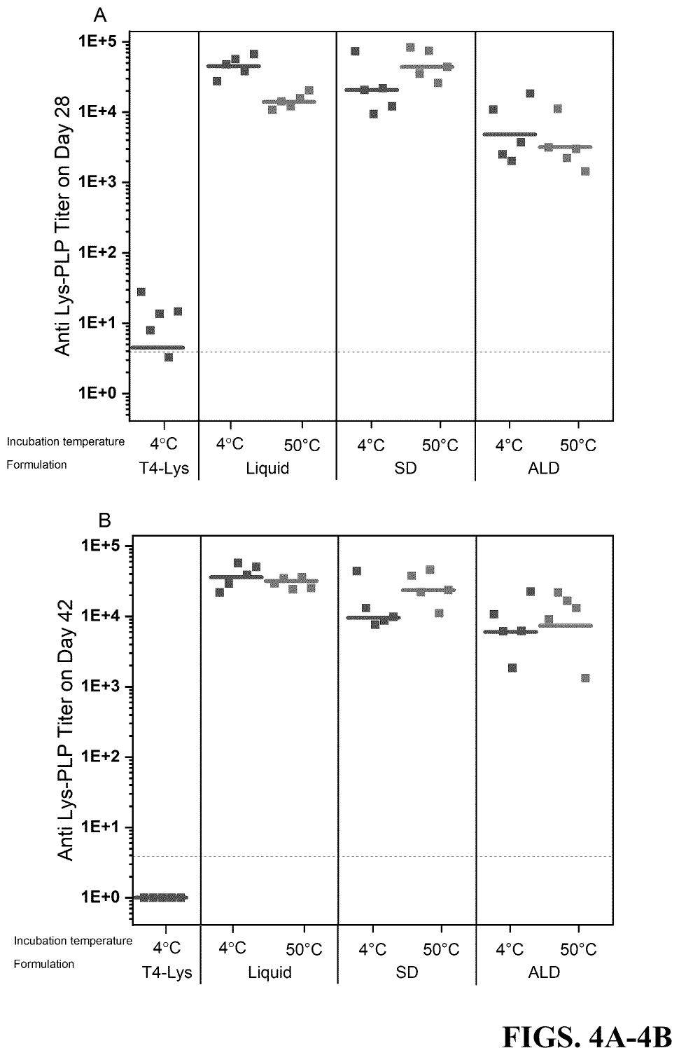

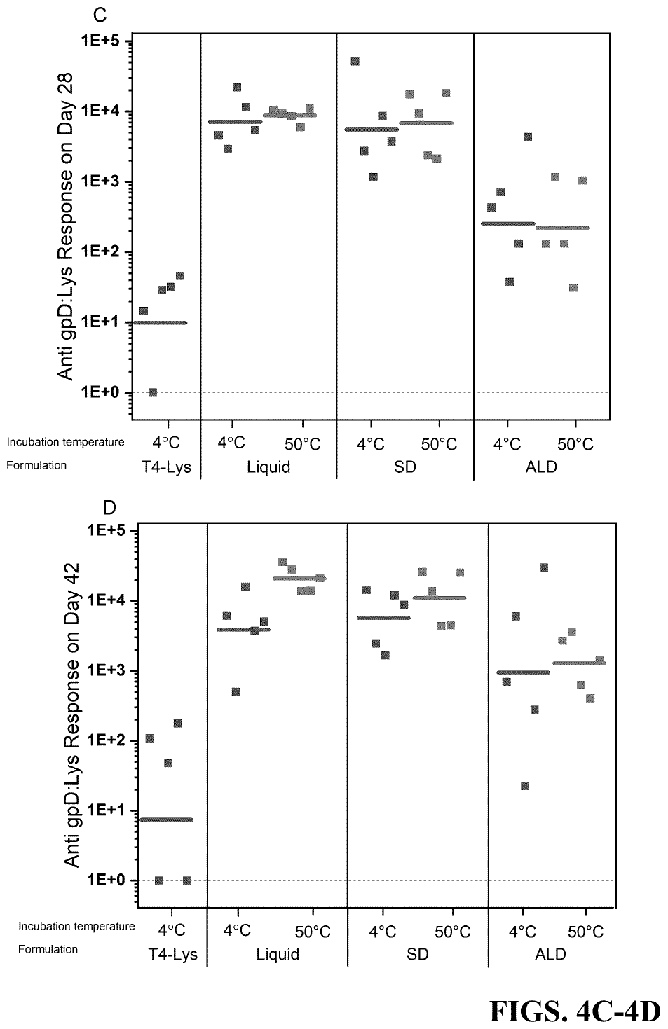

In another exemplary method. immunogenicity of the liquid, spray dried, and ALD formulations after 1 month storage was evaluated in an acceptable animal model (mouse model). About 5 μg doses of liquid decorated PLP (Lys-PLP) formulations that had been incubated for about a month at 4° C. or 50° C. spray dried (SD) Lys-PLPs that had been incubated for about a month at 4° C. or 50° C. or ALD coated Lys-PLPs that had been incubated for about a month at 4° C. or 50° C. were injected into the mice. As a control, one group of mice received 200 ng doses of unmodified agent (T4-lysozyme). equivalent to the amount of T4-lysozyme decorated on the PLPs. Vaccines for all groups except for the groups receiving ALD-coated vaccines included an aluminum adjuvant to boost immunogenicity and were administered in two doses, with a second booster dose given on day 21. The responses from Lys-PLPs (FIGS. 4A and 4B) and T4-lysozyme:gpD (FIGS. 4C and 4D) had similar immunoreactive trends suggesting that antibody responses are directed at the attached T4:lysozyme:gpD antigen and not just to the underlying PLP scaffold.

Interestingly, it was observed that there were minimal immune responses at either time point from the group receiving T4-lysozyme (FIGS. 4C and 4D). This may be due to the small dose (200 ng) of T4-lysozyme administered. In contrast, strong responses against T4 -lysozyme:gpD were observed when equivalent amounts of PLP-conjugated T4-lysozyme were administered to the other groups, highlighting the enhanced immunogenicity resulting from display of the antigen (agent) decorating/loaded onto PLP surfaces.

On day 28, there was approximately half a log difference between the titers in mice administered the liquid and SD formulations that prior to administration had been incubated at 4° C. or 50°0 C. (FIG. 4A). There was a smaller difference on day 28 for the ALD formulation. with a slightly lower mean titer for the group that received ALD-coated vaccines that had been incubated for a month at 50° C. The only statistically significant differences between the titers measured in any of the groups receiving decorated PLP injections (Lys-PLP vaccines) were between those elicited by the SD formulation and the ALD formulations incubated at 50° C. (p≤0.05). Titers on days 28 and 42 were similar for groups that received an initial prime dose and a day 21 booster dose of alum-adjuvanted liquid and SD formulations (FIG. 4B). The immune response in the group that only received one 5 μg dose of ALD-coated vaccine on day 0 was approximately half a log higher on day 42 than on day 28. On day 42, there were no statistically significant differences between the immune responses in any of the groups receiving vaccines containing decorated PLPs (Lys-PLP) (p≥0.05).

FIGS. 4A-4D represent plots depicting antibody responses in mice administered T4-Lysozyme, liquid Lys-PLPs incubated for a month at 4° C. or 50° C. SD Lys-PLPs incubated for a month at 4° C. or 50° C., and ALD coated Lys-PLPs incubated for a month at 4° C. or 50° C. Serum samples were collected on days 28 and 42 after injection, and antibody titers against Lys-PLPs (4A and 4B) and T4-lysozyme:gpD (4C and 4D) were measured by ELISA. The dotted lines indicate the mean titer of all mice on day 0, black horizontal lines indicate the geometric mean titer for each group, and each black square is the mean value of two replicates for one individual mouse. All groups had n=5. On day 28 there was a statistically significant difference between titers generated by the SD formulation that had been incubated at 50° C. and the ALD formulation that had been incubated at 50° C. (p≤0.05). On day 42 there were no statistical differences between anti-Lys-PLP or anti-T4-Lysozyme: gpD titers measured in any of the groups receiving Lys-PLP (p≥0.05).

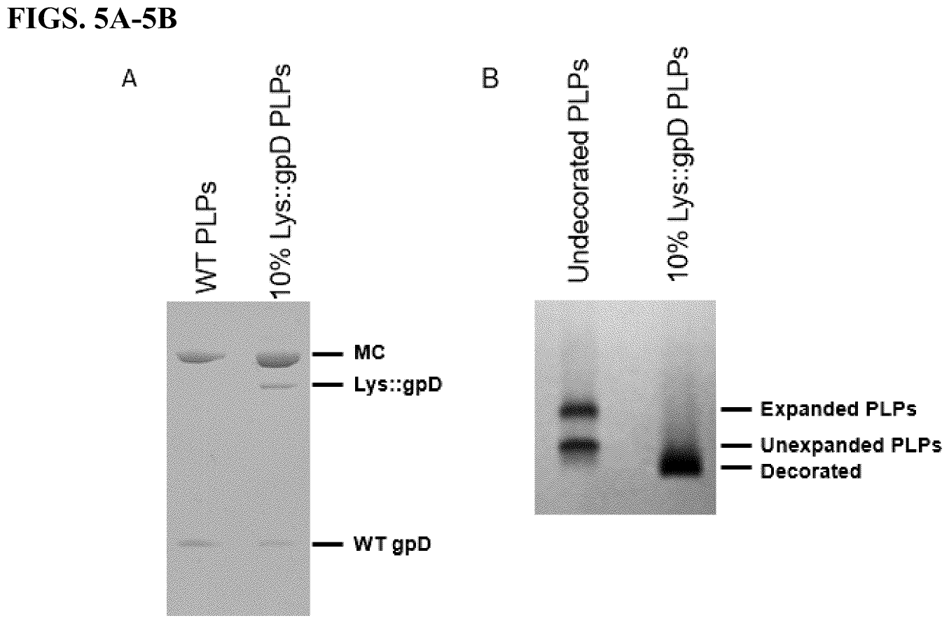

FIGS. 5A-5B. Illustrate gels demonstrating successful decoration of PLPs. 5A illustrates a 12% SDS-PAGE gel demonstrating wild type PLPs (WT) in lane 1 and PLPs decorated with 10% T4-Lysozyme (Lys:gpD) in lane 2. PLPs decorated with T4-lysozyme have an additional band between the WT gpD and major capsid protein (MC) bands. 5B represents an image of a native agarose gel demonstrating undecorated PLPs in lane 1 and PLPs decorated with 10% T4-Lysozyme (Lys:gpD) in lane 2. Undecorated PLPs have two distinct bands whereas decorated PLPs have one larger band.

### Example 4

### Spray Drying of Three Bacteriophage Preparations

In other exemplary methods, phage 80α, phage 2, and phage VPE25 were compared for use as platform phage PLPs in stabilized coated and uncoated decorated forms for uses disclosed herein. In this example, liquid suspensions of phage 80α, phage λ, and phage VPE25 stocks were diluted 20x into an exemplary formulation buffer of 9.5% trehalose. 2.5% hydroxyethyl starch (HES), 10 mM L-Histidine, 40 mM ammonium acetate, and 0.02 mM Tween80 at pH 6.5.

Phage formulations were spray dried in disaccharide-containing formulations using a Buchi B-290 spray dryer (SD). To extract water from the inlet air used as the drying gas in the SD, the air was first fed through a Buchi B-296 dehumidifier. Nitrogen was used as an atomizing gas. The phage formulations were introduced at a flow rate of 0.5 mL/min. The inlet temperature of the drying gas was set to 60° C., resulting in an outlet temperature of approximately 40° C. The aspirator which controlled the drying gas flowrate was set to 60% and the nitrogen atomizing gas was set to 35 mmHg. All systems were run in an open configuration with no nozzle cooling. Spray dried powders were collected via a cyclone separator and aliquoted into 5 mL glass. vials. The resulting powders had a number-weighted particle size of about 4-6 microns.

Vials of the above formulations were placed on the shelf of a LyoStar I lyophilizer. With the shelf temperature held at 20° C., the pressure in the chamber was reduced to 60 mTorr and held for one hour before progressively heating under vacuum to 40° C. Samples were held at 40° C., 60 mTorr for about one hour. Finally, shelf temperature was reduced to 20° C. and the chamber and vials were backfilled to atmospheric pressure with nitrogen. Vials were then capped under nitrogen, crimp-sealed, and removed from the lyophilizer.

### Example 5

### Atomic Layer Deposition Coating of Spray Dried Powders Containing Three Bacteriophage Preparations

Atomic-layer deposition (ALD) was used to deposit thin layers of aluminum oxide (e.g., alumina) on the surface of the spray dried phage particles. Alumina layers were applied be alternating doses of trimethylaluminum (TMA), argon, and water inside a fluidized bed reactor containing the spray dried, phage-containing phage particles.

100 TMA-argon-water cycles were used at a temperature of 50° C. to deposit a total of 100 layers of alumina onto the spray dried particles, resulting in a surface coating of alumina that was approximately 25 nm thick. After coating, samples were stored at 4° C. prior to incubation for stability analysis or biophysical characterization. Particle size distributions of the powders were determined before and after ALD coating using flow imaging microscopy (FIM). Scanning electron microscopy (SEM) was used to qualitatively evaluate SD and ALD-coated phage particle morphologies pre-and post-incubation at varied temperatures. SD powder samples were mounted on imaging stubs with adhesive carbon tape, sputtered with platinum to a coating thickness of 2-3 nm, and imaged on a Hitachi SU3500 Variable Pressure SEM.

### Example 6

### Bacteriophage Activity Assay

In these exemplary experiments, the same protocols were performed to measure the activity of all three phages in both SD and ALD-coated powders. The only variation between protocols for different phages was the use of different host cells since bacteriophage are specific to the bacteria they infect, for example, Phage 80α infects S. Aureus, phage 2, infects E. coli and phage VPE25 infects E. faecalis.

In each case, a single colony of bacteria was picked up and inoculated in 50 ml of LB containing 0.2% maltose with no antibiotics. The suspension culture was grown at 37° C., shaking at 220 rpm, for 8-12 hours, or until the OD₆₀₀ was in the 0.65 to 1.3 range. The bacterial culture was then spun down at 5000 g for 15 minutes. The pellet was resuspended in 20 mL 10 mM MgSO₄ and stored at 4° C. until use.

All steps were performed in a sterile environment. A mechanical uncoating protocol was used to crack open the alumina coatings of the ALD-coated particles prior to infectivity measurement using a plaque assay. Coated phage powders were vortexed for 8 minutes in a 5 mL microcentrifuge tube with a small egg-shaped stir bar. Breaks were taken at 1-minute intervals to tap the tube and prevent the sample from sticking to the sides and lid of the tube. After vortexing, samples were reconstituted in standard media (SM, formulation: 50 mM Tris, 8 mM MgSO₄, 100 mM NaCl, and 0.01% gelatin, pH 7.5 at room temperature) at a ratio of 1 mL to 10 mg of powder sample. Samples were then centrifuged at 500 rpm for 10 minutes at 25° C. to pellet the alumina coating. The phage-containing supernatant was pipetted into a separate tube for use in a plaque assay.

Uncoated phages samples were reconstituted in SM at a ratio of 75.5 μL to 10 mg of powder sample prior to use in the plaque assay. Phage-containing samples were serial diluted with standard media for plating in the plaque assay. Diluted phage samples were mixed 1:1 (100 μL to 100 μL) with the cells they infect (S. Aureus, E. coli, or E. faecalis) and incubated at 37° C. for 20 minutes. Phage-bacteria samples were then mixed with 3 mL top agar (0.55% agar in LB, pre-warmed to 48-50° C.) and poured over plates with bottom agar (1.5% agar in LB) pre-heated to 37° C. Plates were left to incubate for 16-18 hours at 37° C. prior to plaques being counted and conversion to titers (in pfu/mL).

### Example 7

Stability of phage 80α, phage λ, and phage VPE25 in Glassy, Spray-dried trehalose formulations.

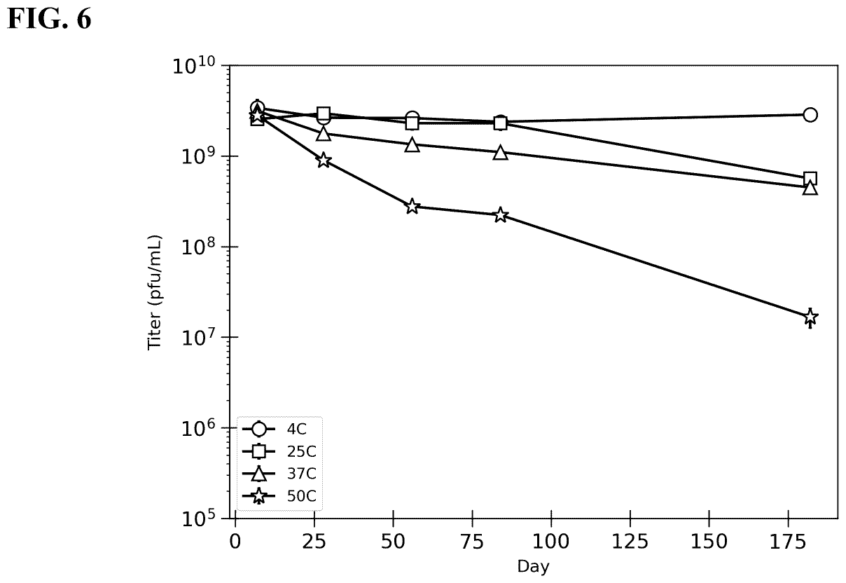

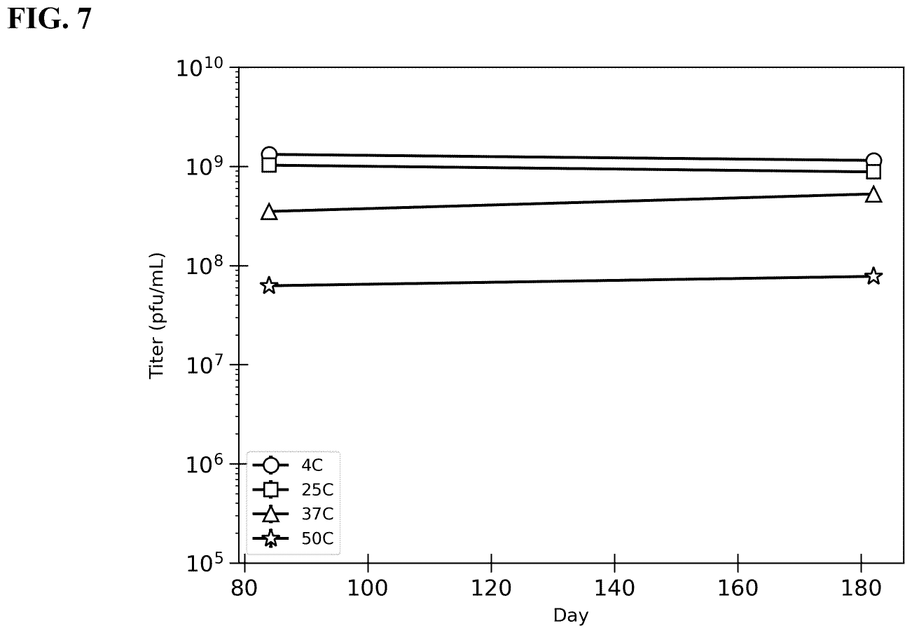

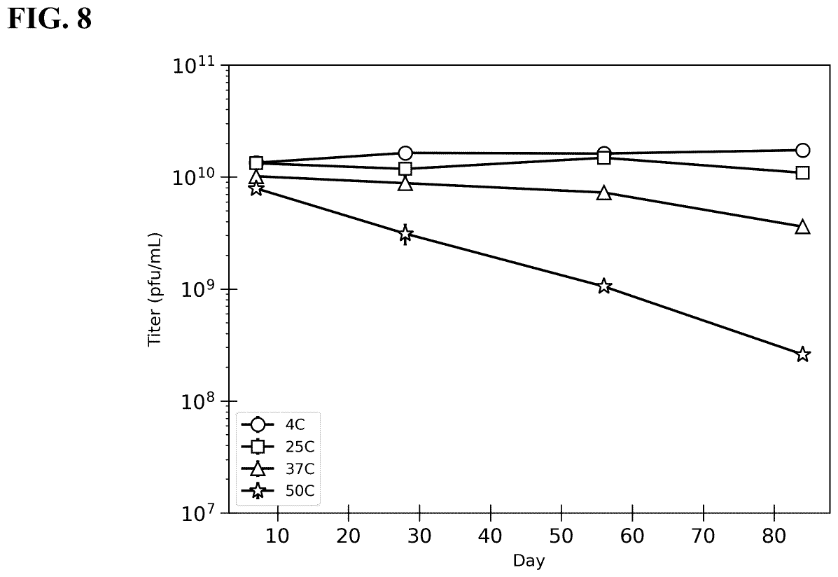

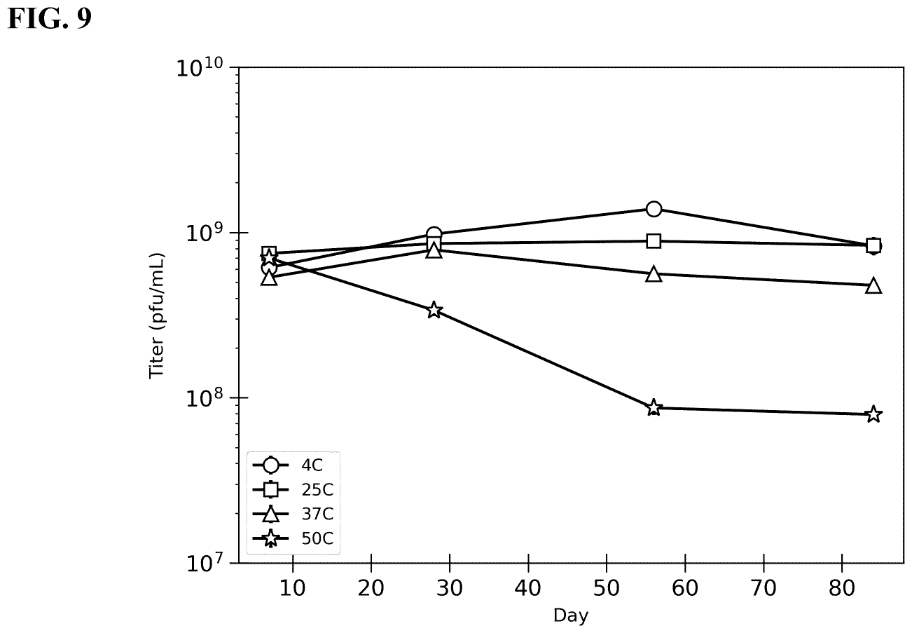

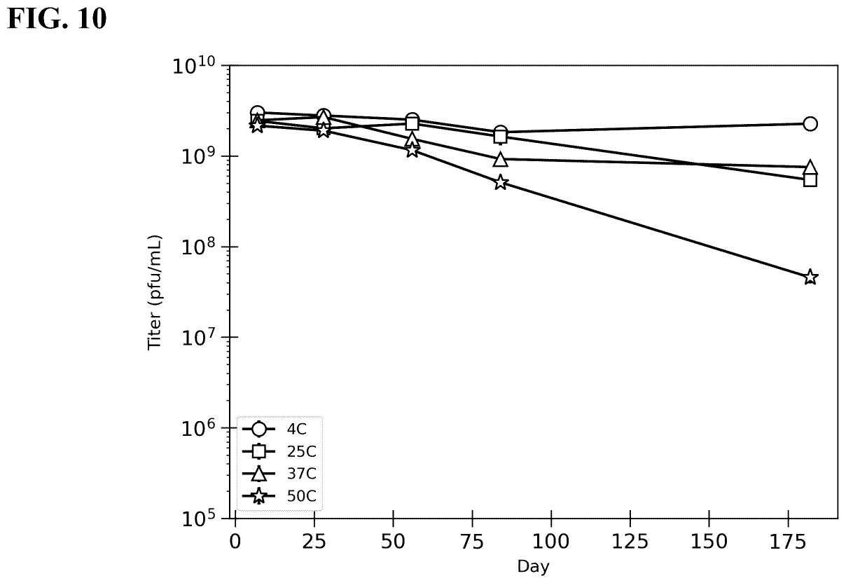

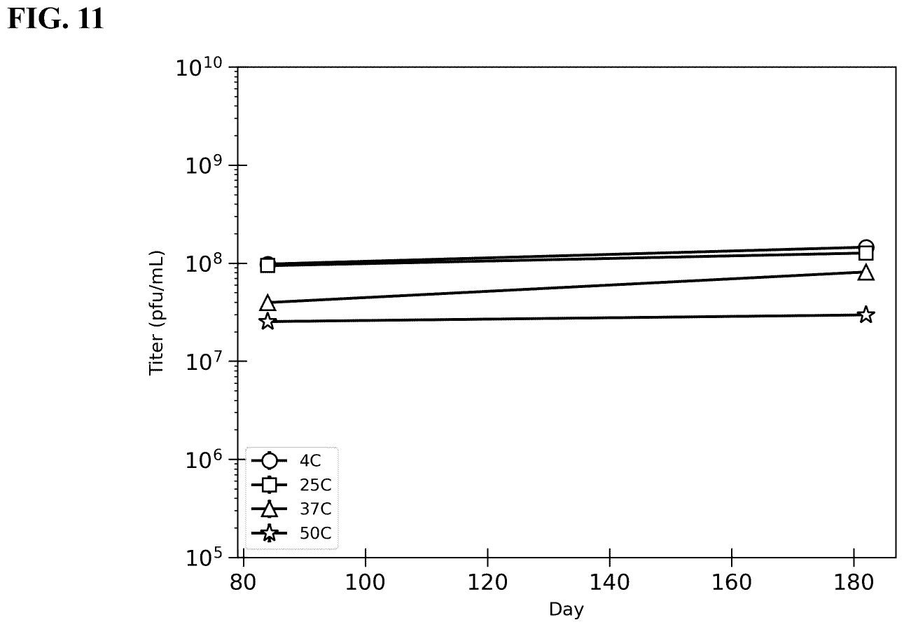

In other exemplary methods, powders prepared according to Examples 4 and 5 above. Phage titers were measured after storage at one of four different temperatures (4, 25, 37, or 50° C.) for up to six months. In both spray dried and spray-dried and ALD-coated powders, phages were remarkably stable, as demonstrated for phage VPE25 (FIGS. 6 and 7), phage 80α (FIGS. 8 and 9) and phage λ (FIGS. 10 and 11).

FIG. 6 illustrates a plot of bacteriophage VPE25 stability in dry. glassy powders prepared by spray drying solutions containing bacteriophage VPE25 in formulations containing 9.5% trehalose, 2.5% hydroxyethyl starch (HES), 10 mM L-Histidine, 40 mM ammonium acetate, and 0.02 mM Tween80 at pH 6.5. Phage activity, expressed as plaque-forming units. was measured against plated cultures of E. faecalis. Powder samples were stored at temperatures of 4. 25. 37. or 50° C. for up to six months. Titer losses less than 1 log were observed after 6 months storage at temperature of 4. 25. and 37° C.

FIG. 7 illustrates a plot of bacteriophage VPE25 stability in dry. glassy powders coated with 100 molecular layers of alumina by ALD. Prior to ALD coating. powders were prepared by spray drying solutions containing bacteriophage VPE25 in formulations containing 9.5% trehalose, 2.5% hydroxyethyl starch (HES), 10 mM L-Histidine, 40 mM ammonium acetate, and 0.02 mM Tween80 at pH 6.5. Phage activity, expressed as plaque-forming units. was measured against plated cultures of E. faecalis. Powder samples were stored at temperatures of 4, 25, 37, or 50° C. and assayed for phage activity after 3 and six months. No titer losses were observed between 3- and 6-months storage at any of the temperatures tested.

FIG. 8 illustrates a plot of bacteriophage 80α stability in dry. glassy powders prepared by spray drying solutions containing bacteriophage 80α in formulations containing 9.5% trehalose, 2.5% hydroxyethyl starch (HES), 10 mM L-Histidine, 40 mM ammonium acetate. and 0.02 mM Tween80 at pH 6.5. Phage activity, expressed as plaque-forming units. was measured against plated cultures of S. aureus Powder samples were stored at temperatures of 4, 25, 37, or 50° C. for up to six months. Titer losses less than 1 log were observed after 6 months storage at temperature of 4, 25, and 37° C.

FIG. 9 illustrates a plot of bacteriophage 80α stability in dry, glassy powders coated with 100 molecular layers of alumina by ALD. Prior to ALD coating, powders were prepared by spray drying solutions containing bacteriophage 80% in formulations containing 9.5% trehalose. 2.5% hydroxyethyl starch (HES), 10 mM L-Histidine. 40 mM ammonium acetate. and 0.02 mM Tween80 at pH 6.5. Phage activity, expressed as plaque-forming units. was measured against plated cultures of S. aureus. Powder samples were stored at temperatures of 4, 25, 37, or 50° C. and assayed for phage activity at various time points for up to six months. No titer losses were observed for samples stored at 4. 25. or 37° C.: samples stored at 50° C. demonstrated less than a 1-log loss of activity against S. aureus cultures.

FIG. 10 illustrates a plot of bacteriophage λ stability in dry. glassy powders prepared by spray drying solutions containing bacteriophage λ in formulations containing 9.5% trehalose, 2.5% hydroxyethyl starch (HES), 10 mM L-Histidine, 40 mM ammonium acetate. and 0.02 mM Tween80 at pH 6.5. Phage activity, expressed as plaque-forming units. was measured against plated cultures of E. coli. Powder samples were stored at temperatures of 4, 25, 37, or 50° C. for up to six months. Titer losses less than 1 log were observed after 6 months storage at temperature of 4, 25, and 37°0 C.

FIG. 11 illustrates a plot of bacteriophage λ stability in dry, glassy powders coated with 100 molecular layers of alumina by ALD. Prior to ALD coating, powders were prepared by spray drying solutions containing bacteriophage 2. in formulations containing 9.5% trehalose, 2.5% hydroxyethyl starch (HES), 10 mM L-Histidine, 40 mM ammonium acetate. and 0.02 mM Tween80 at pH 6.5. Phage activity, expressed as plaque-forming units, was measured against plated cultures of E. coli. Powder samples were stored at temperatures of 4. 25, 37, or 50° C. and assayed for phage activity after 3 and six months. No titer losses were observed between 3- and 6-months storage at any of the temperatures tested.

In these proof-of-concept studies of Examples 1-7 above, utility of a single-shot stabilization technology in two studies was evaluated. In the first study, retention of phage infectivity after spray drying and storage at 37° C. as a sensitive indicator of the stability of the phage platform underlying the Lys-PLP vaccine. Phage therapy has received increasing attention as an alternative and/or adjuvant therapy in the setting of multi-drug resistant bacterial infections; however, several issues arise in the application of phage therapy as a mainstream therapeutic approach. These include delivery, distribution, and regulatory hurdles. Development of pharmaceutically-relevant formulations of phages that confer adequate stability for long-term storage remains a major challenge. While liquid formulations of phage were stable at room temperature, the same formulations lost almost 8 logs of infectivity after being incubated at 37° C. for nine months. In contrast, spray dried phage showed no loss of infectious activity even after a year of incubation at 37° C. Previous studies have successfully thermostabilized phages using spray drying, however, were limited to incubation at room temperature. Therefore. the data presented herein demonstrate that these processes stabilize infectious phages for extended time periods at elevated temperatures. Moreover, the formulated powders can be rapidly reconstituted with water, further simplifying the procedures for phage delivery in the field.

Even without ALD coating, the thermostabilization of phage could be of benefit for many other applications. While currently there are no approved phage therapeutics, phages are used in food production. Additionally, phage therapy has the potential to improve treatment options for bone infections after injury or reconstructive surgery. These infections contribute to ongoing illness after surgery and reconstruction and may ultimately result in treatment failure and other detrimental side effects.

Further, it was observed that decorating the surface of phage-based PLPs with a model antigen, T4-lysozyme:gpd was successfully stabilized and induced a response in coated and uncoated form after prolonged incubation at elevated temperatures (FIGS. 4A-4D). Here. the ability of ALD-coated, spray dried powders to create controlled-release therapeutic agent decorated preparations that combine prime and boost doses in a single shot were assessed. Single doses of decorated PLPs (Lys-PLP) injections that were coated with for example, 250, molecular layers of alumina elicited antibody responses that six weeks following administration were equivalent to responses generated by two separate doses of conventional formulations. Further, the titers were unaffected by prolonged storage of the vaccines at 50° C.

In this study, the immune responses detected on day 42 after a single injection of ALD coated formulations were similar to the responses generated by two doses of adjuvanted liquid and spray dried decorated PLP (Lys-PLP) formulations. Even though TEM analysis of Lys-PLPs in the liquid formulation incubated at 50° C. suggest that the PLPs were somewhat aggregated, the formulation still elicited a strong immune response. This is in contrast to the minimal anti-Lys-PLP and anti-T4-Lysozyme:gpD responses exhibited in the group administered soluble T4-lysozyme, as demonstrated in these ELISA assays.

One key advantage of this decorated PLP platform is that antigens are conjugated to specific decoration sites on the PLP capsid surface, in reactions that are conducted in vitro. In these studies. PLPs and the decoration antigen T4-Lysozyme:gpD were synthesized and purified separately before being linked in vitro via the gpD. A wide variety of molecules can be coupled using the gpD protein to the PLP surface without the need to genetically modify the PLPs or change the processes by which they are synthesized and purified, offering flexibility in modifications to the PLPs surface. For example, to adapt a PLP-based composition to meet changing needs such as the appearance of a pathogen variant. a modified antigen can simply be attached in vitro to standard PLP surfaces. Furthermore, the use of ALD coated PLPs eliminates the need for an additional booster requiring only a single dose that is as efficacious as conventional multi-dose schemes.

Demonstrated herein are PLP designer particle constructs as a feasible multifaceted vaccine or therapeutic candidate that is stable for prolonged periods at 5020 C. after spray drying, and spray drying and ALD. The development of this platform allows for the potential distribution worldwide to resource poor areas that lack the infrastructure to distribute vaccines that require a cold chain. Furthermore, it has been demonstrated that it is possible to thermostabilize any phage including. but not limited to, phage λ. phage VPE25, and phage 80α, each can be used as potential antibiotic alternatives active against infections of E. coli, E. faecalis, and S. aureus, respectively.

### Materials and Methods

### Materials

The alum adjuvant, Alhydroge®, was purchased (e.g., Accurate Chemicals and Scientific Corp (Westbury, NY)). Buffer components used to prepare spray dried powders were purchased from a variety of sources: high purity trehalose was purchased from Pfanstiehl (Waukegan, IL), hydroxyethyl starch (HES) was from Fresenius Kabi (Bad Homburg, Germany), Ammonium acetate was from J. T. Baker (Avantor, Radnor, PA), trimethylaluminum (TMA), histidine and borate were from Sigma-Aldrich (St. Louis, MO). Urea, HEPES, L-arginine hydrochloride, ethylenediaminetetraacetic acid, sodium phosphate, sodium citrate, sodium chloride, polysorbate 80, polysorbate 20, boric acid, phosphate buffered saline, and sulfuric acid were all purchased from Thermo Fisher Scientific (Waltham, MA). Monosodium L-Glutamate was from Spectrum Chemical Mfg. Corp (New Brunswick, NJ). Powdered milk was purchased from Safeway (Pleasanton, CA). Goat anti-mouse HRP secondary antibody was purchased from Promega (Madison, WI). SureBlue TMB substrate from SeraCare (Milford, MA). Nominal 10 mL type I borosilicate glass vials, butyl rubber stoppers, and aluminum seals from DWK Life Sciences (Millville, NJ) were used for storage of the vaccine formulations. Sepharose columns, HiTrap Q wash buffer and columns were from Cytiva (Marlborough, MA)

### Phage λ Purification and Infectivity Assay

In one example, bacteriophage λ (e.g., cI858 Sam7) was purified using a published procedure and phage titers were quantified using Escherichia coli LE392 as the host in a standard plaque assay to measure activity.

### Phage-Like Particle (PLP) Expression and Purification

In another example, PLPs were expressed and purified as previously described. In brief, BL21(DE3) E. coli cells were transformed with pNu3_E plasmid that expresses the phage A major capsid (gpE) and scaffolding (gpNu3) proteins that self-assemble into PLPs. Any other phage known in the art could be used for these examples (e.g., phage 80α, phage VPE25, etc.) single colony was isolated to inoculate Lauria broth (10 ml) containing 100 μg/mL ampicillin and the culture was incubated at room temperature overnight with shaking at 220 rpm. The 10 mL culture was then added to IL terrific broth containing 50 μg/mL ampicillin and allowed to grow at 37° C. until the OD₆₀₀ reached 0.6. 1 mM Isopropylthio-β-galactoside (IPTG) was then added, the culture was maintained for an additional two hours with shaking, and the cells then harvested by centrifugation to form a cell pellet which was collected after decanting the supernatant. Unless otherwise indicated, all subsequent steps were performed at 0-4° C.

Following the above example, the cell pellet was resuspended in ice-cold TMS buffer (25 ml, 50 mM Tris buffer, pH 8 at 4° C. containing 100 mM NaCl, 10 mM MgCl2), the cells were lysed by sonication and the crude lysate was clarified by centrifugation (7.650×g, 10 minutes). The supernatant was spun at high speed (97.000×g. 3 hours) and the pellet, which contained the PLPs. was overlaid with TMS buffer (1 ml) and allowed to incubate overnight. The supernatant was collected, overlayed on a 10-40% sucrose gradient in the same buffer and centrifuged at 97.000×g for 3 hours. The PLP band (located roughly in the center of the gradient) was visualized with ambient light and harvested by aspiration. The sample was diluted 2-fold with HiTrap Q wash buffer (20 mM Tris buffer, pH 8 at 4°0 C. containing 15 mM magnesium chloride, 1 mM EDTA, 7 mM-mercaptoethanol), loaded onto a 5 mL HiTrap Q column equilibrated with HiTrap Q Wash buffer, washed with 5 column volumes, and eluted with a 10-column volume linear gradient to 1 M NaCl. Fractions (5 mL) were collected, and PLP-containing fractions pooled and concentrated using a 100 kDa MWCO filter. Then, PLPs were dialyzed into 50 mM HEPES buffer. pH 7.4. containing 100 mM sodium chloride, 10 mM magnesium chloride. The concentration of purified PLPs was determined spectrally (ε280)=16.32 μM-1 cm−1) and stored at 4° C. until further use.

### T4-Lysozyme and T4-Lysozyme:gpD Expression and Purification

In another example, E. coli BL21 (DE3) cells were transformed with an exemplary agent, T4-lysozyme or a T4-lysozyme:gpD (Lyso:D). fusion plasmid and subsequently plated on an LB plate supplemented with 100 μg/mL ampicillin overnight at 3720 C. A single colony was isolated and used to inoculate 10 mL of LB media supplemented with 100 μg/mL ampicillin. After overnight incubation, the 10 mL culture was used to inoculate 1 L of TB media supplemented with 50 μg/mL ampicillin, and the culture was allowed to incubate at 37° C. with 200 rpm shaking until the OD₆₀₀ was between 0.6 and 0.8. The culture was then induced by the addition of IPTG to a final concentration of 1 mM and allowed to incubate for an additional 2 hours at 30° C. with 200 rpm shaking. The cell pellet was harvested by centrifugation at 5.000×g for 15 minutes and the cell pellet was resuspended in 10 mM sodium phosphate, 20 mM sodium chloride, 1 mM magnesium chloride, 0.1 mM calcium chloride, 50) mM EDTA, pH 6.6 supplemented with a Pierce Protease Inhibitor Tablet (ThermoFisher). Cells were lysed by sonication on ice and centrifuged at 13000×g for 30 minutes at 4° C. The supernatant was collected and loaded onto a HiTrap SP HP 5 mL column (Cytiva) equilibrated with a wash buffer 50 mM tris, 1 mM EDTA, 50 mM sodium chloride, pH 7.25. The sample was washed with 5 column volumes of the wash buffer and eluted with a 10-column volume linear gradient of an elution buffer containing 50 mM tris, 1 mM EDTA, 50 mM sodium chloride, 1 M sodium chloride, pH 7.25). T4-lysozyme or Lyso:D-containing fractions were pooled and concentrated to ≤5 mL and loaded onto a HiPrep 16/60 Sephacryl S-100 HR column (Cytiva) equilibrated with Lysozyme storage buffer (10 mM borate, 40 mM ammonium acetate. 10% glycerol, pH 8) at a flow rate of 0.5 mL/min. T4-lysozyme or Lyso:D-containing fractions were pooled and concentrated using a 10 kDa MWCO filter and concentration was measured by absorbance spectroscopy (T4-Lysozyme ε280=25440 M-1·cm−1; Lyso:D ε280 =40910 M-1·cm−1). Samples were stored at −20° C. until use.

Decoration or Loading of PLPs with an Exemplary Agent, T4-Lysozyme

In this example, PLPs were buffer exchanged into ice-cold 2.5 M urea, 0.01% (w/v) polysorbate-20, 10 mM HEPES, pH 7.4, and allowed to incubate for 1 hour to induce expansion. Next, PLPs were buffer exchanged into ice-cold 0.2 M urea, 0.01% (w/v) polysorbate-20, 10 mM HEPES, pH 7.4, and diluted to 20 nM. The decoration proteins were added to the PLPs such that Lyso: gpD would constitute approximately 10% of the total surface density: gpD was used to make up the remaining 90% of the surface density. Samples were allowed to incubate at room temperature for about 1 hour to create fully decorated PLPs (Lys-PLPs). A Superose 6 Increase 10/300 GL column (Cytiva) was equilibrated with borate buffer (10) mM borate, 40 mM ammonium acetate, 10% glycerol. pH 8). The decorated PLPs were injected onto the column at a flow rate of 0.5 mL/minute, and PLP-containing fractions were pooled and concentrated using a 100 kDa MWCO filter. PLP concentration (100.5 nM) was measured by absorbance (ε280=23.88 μM-1·cm−1) and samples were stored at 4° C. until further use. Successful decoration with T4-Lysozyme was confirmed with SDS-PAGE and native agarose gels (Supplemental FIG. 1).

### Spray Drying of Lys-PLPs (Decorated PLPs)

In this example. decorated PLPs (e.g., Lys-PLPs) were suspended in a buffer of 9.5% trehalose (glass-forming agent), 2.5% hydroxyethyl starch (HES), 0.02 mM Tween80, 40 mM ammonium acetate, and 10 mM borate, pH 8 to a final concentration of 0.1 mg/mL. The formulation was spray dried using a Buchi B290 spray dryer (New Castle, DE). The inlet temperature was set to 110° C. the aspirator to 50%, and nitrogen drying gas flowed at a rate of 50 L/hr with an in-line dehumidifier. The resulting outlet temperature varied between about 66° C. and about 67° C. After spray drying was completed. the formulation was further dried overnight using a LyoStar I lyophilizer from FTS Systems (Warminster, PA) with the shelf temperature set to 40° C. and vacuum to 60 mTorr. After further drying, the vials were backfilled with nitrogen and capped. Moisture levels in the spray dried powders were determined by Karl-Fischer analysis. The residual water content of the spray dried powders was ˜1%; further drying reduced moisture contents of the powders below 1% (data not shown). At these low moisture contents, glass transition temperatures (Tg's) are expected to be substantially above any anticipated storage condition, as previously reported where it was observed that similar formulations containing 9.5% trehalose had Tg's between 106° C. and 110° C., and that addition of hydroxyethyl starch further increased Tg's by 15-20° C.

### Atomic Layer Deposition Coating of Spray Dried Particles

In another exemplary methods, decorated PLP particles can be coated. For example, using alternating gas-phase injections of trimethylaluminum and water in a custom-built atomic layer deposition (ALD) fluidized bed reactor, spray dried powders were coated with metal oxide. In this example, 250 molecular layers of Al₂O₃ as previously described were created encapsulating the decorated PLP essentially dry particles. Al₂O₃ contents of the ALD-coated powders were estimated by weight loss after high temperature calcining to remove volatilizable excipients to be about 19.5%, corresponding to an Al₂O₃ layer thickness on each particle of about 60 nm. After coating, particles were stored at 4° C. prior to incubation studies.

Incubation study

In another example, liquid, spray dried, and spray dried-ALD coated formulations were incubated at 4° C. or 50° C. for about one month to assess stability of coated particles against control samples of liquid or just spray dried uncoated particles. Samples were analyzed before and after incubation by electron microscopy and light scattering.

### Phage Study

In these examples, Phage λ was used as a representative phage and suspended in 9.5% trehalose, 2.5% HES, 0.02 mM Tween80, 50 mM Tris, and 8 mM MgSO₄. The formulation was spray dried as described above. Both spray dried and liquid formulations were incubated at room temperature or about 37° C. for up to one year. A plaque assay was then performed to test the infectivity of the virus. Briefly, a single colony of E. coli LE392 was collected and grown in LB with 0.2% maltose at 37° C. with shaking at 220 rpm for at least 8 hours. The culture was then centrifuged at 5,000×g for 15 minutes and resuspended in 10 mM MgSO₄. Phage was then diluted 102, 104, 106, 107 and 108 in SM solution (50 mM Tris, pH 7.5, 8 mM MgSO4, 100 mM NaCl, and 0.01% gelatin) at room temperature. 100 μL of diluted phage and SM solution were mixed with 100 μL of E Coli and incubated at 37° C. for 20 minutes. After incubation the mixture was added to 3 mL of 0.7% agar in LB at 50° C. mixed. and added to an LB plate and grown overnight at 37° C. The plaques were counted the next day and the plaque forming units (pfu) per mL were determined. Each experiment was performed in at least duplicate with no more than 20% difference between each replicate.

### Assessment of Particle Size Using Dynamic Light Scattering (DLS)

In another method, to probe the aggregation state of decorated/loaded PLPs (Lys-PLPs). the hydrodynamic diameters of the Lys-PLPs were analyzed in various buffers before stabilization and after incubation using an Anton Paar Litesizer 500 (Graz, Austria). 100 μL of sample was placed in an Eppendorf UVette disposable cuvette (Hamburg, Germany) and analyzed using side-scatter (175°. To analyze whether aggregation of Lys-PLPs occurred within ALD-coated particles. DLS analyses of PLPs were conducted after the deposited alumina coatings were removed by dissolving them in 60 mM sodium phosphate, 100 mM sodium citrate, 150 mM sodium chloride, 0.04% polysorbate-80. pH 6.7 buffer.

### Transmission Electron Microscopy (TEM)

Transmission electron microscopy was used as another method to characterize the size and morphology of decorated PLPs (Lys-PLPs) within the various formulations. Samples were analyzed both prior to and after incubation, samples were imaged using transmission electron microscopy (TEM). Spray dried powders were reconstituted by addition of purified water, in which they dissolved within a few minutes. In contrast, ALD-coated powders (spray dried, decorated PLPs that were ALD coated) are insoluble in water. Therefore, to image the PLPs contained within the ALD-coated powders they were suspended in 60 mM sodium phosphate, 100 mM sodium citrate, 150 mM sodium chloride, 0.04% polysorbate-80. pH 6.7which dissolved the alumina coating and released the PLPs from the inner core of the particles into the suspending medium. Dissolution of the coatings was complete within 24 hours, after which time samples were adhered to 400 mesh carbon coated grids and stained using 2% uranyl acetate. Grids were imaged on a Tecnai T12 Spirit Biotwin TEM (Hillsboro, OR) operating at 100 kV. fitted with an AMT CCD (Woburn, MA).

### Scanning Electron Microscopy (SEM)

In another example, to evaluate particle morphology and size of spray dried and ALD coated particles prior to and after incubation, samples were imaged using scanning electron microscopy (SEM). Dry powders were mounted on imaging stubs using adhesive carbon tape and sputtered with platinum for 15 seconds using a 108 Auto Sputter Coater from Cressington (Liverpool, UK). Stubs were imaged on a Hitachi SU3500 Variable Pressure SEM (Hitachi).

### Immunogenicity Testing in BALB/C Mice

In another exemplary method, female BALB/c mice were purchased from Taconic Biosciences (Hudson, NY) at 5-6 weeks old. 5 animals per group were used. Animals were injected intramuscularly with 5 μg doses of liquid Lys-PLP incubated at 4° C. or 50° C. spray dried (SD) Lys-PLPs incubated at 4° C. or 50° C. or ALD coated Lys-PLPs incubated at 4° C. or 50° C. An additional group of five animals received a dose of agent (T4-lysozyme) equivalent to the amount of lysozyme decorated on the PLPs (0.187 μg). All formulations. except the ALD formulations received an additional booster dose on day 21. The T4-lysozyme and liquid Lys-PLP formulations were prepared in the same buffer used for spray drying, along with the adjuvant Alhydrogel® (0.1 mg/mL). Spray dried formulations were reconstituted in 0.1 mg/mL Alhydrogel R in water for injection. Samples with powders coated with ALD-deposited alumina were resuspended in 9.5% trehalose, 10 mM L-histidine. pH 6 to prevent settling of the coated particles, but did not have added Alhydrogel. Blood was collected on day 0) and 28 via submandibular bleeds. A terminal bleed on day 42 was collected via cardiac puncture. All serum was stored at −80° C. until use.

In another exemplary method, to assess the anti-agent/cargo PLP (e.g., anti-Lys-PLP) response. Immulon4HBX 96-well plates from ThermoFisher Scientific (Waltham, MA) were coated with 0.005 mg/mL Lys-PLPs or 0.01 mg/mL T4-lysozyme:gpD and placed on a rocker plate at 4° C. overnight. Plates were washed five times with a wash buffer consisting of 0.04% polysorbate 20 in phosphate buffered saline in a BioTek plate washer (Winooski, VT) and then blocked with 3% cow's milk for 1 hour at 37° C. Plates were washed five times. 3% cow's milk was added to all wells on the plate, sera was added to the first well of each row, and diluted across the plate. The plate was incubated for 1 hour at 37° C. washed five times, and a 1:10.000 dilution of goat anti-mouse HRP secondary antibody in 3% milk added to each well. The plate was incubated for one hour at 37°0 C. washed 5 times, and SureBlueTM TMB was added to each well. The reaction was stopped with 0.5 M sulfuric acid and the absorbance was read on a Tecan Microplate (Männedorf. Switzerland) at 450 nm. All ELISA results were completed in duplicate and anti-Lys-PLP and anti-T4-lysozyme:gpD titers for each animal were calculated by fitting to using a 4-parameter logistic equation as previously described.

### Statistical Analysis

All statistical analysis was completed using OriginPro. Version 2021 (Northampton, MA). The normality of the data was tested using a Shapiro-Wilk test. Normality was rejected, and to determine if there was a statically significant difference in anti-Lys-PLP titers a Kruskal-Wallis ANOVA with Dunn's post-hoc test was performed. A p-value ≤0.05 was considered significant.

All of the compositions and methods disclosed and claimed herein can be made and executed without undue experimentation in light of the present disclosure. While the compositions and methods have been described in terms of embodiments, it is apparent to those of skill in the art that variations maybe applied to the compositions and methods and in the steps or in the sequence of steps of the methods described herein without departing from the concept, spirit and scope herein. More specifically, certain agents that are both chemically and physiologically related may be substituted for the agents described herein while the same or similar results would be achieved. All such similar substitutes and modifications apparent to those skilled in the art are deemed to be within the spirit, scope and concept as defined by the appended claims.

What is claimed is:

1. A thermostable phage-containing particle formulation comprising:
a composition comprising:
a phage or phage like particle (PLP);
at least one glass-forming agent;
at least one smoothing agent;
at least one volatile salt; and
at least one buffer.

2. The formulation according to claim 1, wherein the composition is essentially dry and forms essentially dry particles.

3. The formulation according to claim 1, wherein the at least one glass-forming agent comprises at least one of trehalose, sucrose, ficoll, dextran, sucrose, maltotriose, lactose, mannitol, glycine, maltose, lactulose, mannitol and glycine, cyclodextrin, povidone, or combinations thereof.

4. The formulation according to claim 1, wherein the at least one smoothing agent comprises at least one of hydroxyethyl starch (HES), polyvinyl pyrrolidone, chitosan, human serum albumin (HSA), other serum albumins, dextran, hetastarch, plasma protein factor, or combinations thereof.

5. The formulation according to claim 1, wherein the at least one volatile salt comprises at least one of ammonium acetate, ammonium formate, ammonium carbonate, ammonium bicarbonate, triethylammonium acetate, triethylammonium formate, triethylammonium carbonate, trimethylamine acetate, trimethylamine formate, trimethylamine carbonate, pyridinal acetate and pyridinal formate, or combinations thereof.

6. The formulation according to claim 1, wherein the formulation further comprises at least one surfactant and the at least one surfactant comprises at least one non-ionic surfactant.

7. The formulation according to claim 6, wherein the at least one surfactant comprises at least one polysorbate comprising at least one of Tween 20,Tween 40, Tween 60, and Tween 80, or at least one poloxamer comprising at least one of poloxamer 188, poloxamer 407, poloxamer 235, and poloxamer 335, or Brij, or alkylphenol hydroxypolyethylene surfactants such as comprising at least one of Triton X100, Triton X114 and Triton X405, or oligoethylene glycol monoalkyl ethers or combinations thereof.

8. The formulation according to claim 1, wherein the buffer comprises at least one of histidine, glycine, arginine, phosphate, citrate, acetate, Tris (Hydroxymethyl) aminomethane, succinate, prolamine, borate, carbonate, or a combination thereof.

9. The formulation according to claim 6, wherein the at least one disaccharide comprises trehalose, the at least one smoothing agent comprises HES, the at least one surfactant comprises Tween80, the at least one volatile salt comprises ammonium acetate and the at least one buffer comprises histidine.

10. The formulation according to claim 1, wherein the phage or phage like particle (PLP) further comprises at least one decoration permitting linking agent or decoration linking agent.

11. The formulation according to claim 10, wherein the at least one decoration permitting linking agent or decoration linking agent comprises gpD (phage λ capsid decoration protein gpD).

12. The formulation according to claim 10, wherein the phage or PLP having the at least one decoration permitting linking agent or decoration linking agent further comprises at least one therapeutic agent, biologic, or antigen linked to or bound to, the at least one decoration permitting linking agent or decoration linking agent.

13. The formulation according to claim 12, wherein the therapeutic agent, biologic, or antigen comprises at least one of a viral antigen, a bacterial antigen, a toxin, a prion, yeast, a biologically-relevant fragment, or subunit thereof.

14. The formulation according to claim 12, wherein the therapeutic agent comprises at least one of a chemical agent, a small molecule, an anti-cancer agent, an anti-inflammatory agent, an anti-autoimmune agent, an antibody or fragment thereof, a peptide, a protein, or a combination thereof.

15. The formulation according to claim 12, wherein the therapeutic agent, biologic, or antigen comprises at least one of a recombinant peptide, a recombinant protein, a peptide derived from a target protein or pathogen, a polynucleotide, a polynucleotide encoding a polypeptide, a polysaccharide derived from a target pathogen, a virus-like particle, a live virus, a live, attenuated virus, an inactivated virus, an antigen attached to, associated with, or expressed on the surface of a virus or a combination thereof.

16-18. (canceled)

19. The formulation according to claim 2, wherein the composition is essentially dry and further comprising introducing one or more coating layers of at least one of metallo-organic material, metal oxide or metal alkoxide using atomic layer deposition (ALD) and essentially completely covering the essentially dry particles.

20. (canceled)

21. A composition comprising a plurality of the particles according to claim 2; optionally further comprising a pharmaceutically acceptable excipient.

22-24. (canceled)

25. A method of making a stabilized phage particle or PLP according to claim 1, the method comprising:
a) combining phage or PLP with at least one disaccharide agent; at least one smoothing agent; at least one volatile salt; at least one surfactant; and at least one buffer in a liquid composition; and
b) spray-drying a) to make essentially dry phage particles or essentially dry PLPs.

26. (canceled)

27. The method according to claim 25, further comprising introducing at least one of a decoration permitting linking agent or decoration linking agent or loading the phage particles or PLPs in a) with at least one therapeutic agent, biologic, or antigen.

28-34. (canceled)

35. A method for treating, reducing onset of, and/or preventing a pathogenic microorganism infection in a subject comprising, administering to the subject a composition according to claim 21, and treating, reducing onset of, and/or preventing the pathogenic microorganism infection in the subject.

36. The method according to claim 35, wherein the pathogenic microorganism comprises a pathogenic bacteria and the phage or PLPs comprises at least one of undecorated phage, decorated phage, undecorated PLPs, or decorated PLPs in the composition.

37-38. (canceled)

39. A method for treating, ameliorating, and/or reducing the risk of onset of a health condition in a subject, the method comprising administering to the subject a composition according to claim 21, and treating, reducing onset of, and/or ameliorating the health condition in the subject.

40-41. (canceled)

42. A kit comprising at least one composition according to claim 2, and at least one container.
